(19) United States
(12) Patent Application Publication
(10) Pub. No.: US 2012/0122870 A1
(43) Pub. Date: May 17, 2012

(54) Treatment Of Ischemia-Reperfusion Injury

(75) Inventors: Charles D. Smith, Mount Pleasant, SC (US); Zhi Zhong, Charleston, SC (US); Lynn W. Maines, Hummelston, PA (US)

(73) Assignees: MUSC FOUNDATION FOR RESEARCH DEVELOPMENT, Charleston, SC (US); APOGEE BIOTECHNOLOGY CORPORATION, Hummelstown, PA (US)

(21) Appl. No.: 13/319,392

(22) PCT Filed: May 10, 2010
(86) PCT No.: PCT/US10/34239

§ 371 Date: Jan. 27, 2012

Related U.S. Application Data
(60) Provisional application No. 61/176,636, filed on May 8, 2009.
(60) Provisional application No. 61/229,272, filed on Jul. 28, 2009.

(51) Int. Cl. A61K 31/165; A61K 31/222; A61K 31/36; A61K 31/4468; A61K 31/495; A61K 31/40; A61K 31/4015; A61K 31/5375; A61K 31/4409; A61K 31/4164; A61K 31/404; A61K 31/41; A61K 31/426; A61K 31/428; A61K 31/423; A61K 31/52; A61K 31/135; A61K 31/136; A61K 31/192; A61K 31/381; A61K 31/427; A61K 31/4439; A61K 31/496; A61K 31/5355; A61P 9/10; A61K 31/12; A61K 31/167

(52) U.S. Cl. 514/236.8; 514/617; 514/623; 514/511; 514/466; 514/325; 514/255.01; 514/428; 514/424; 514/237.8; 514/357; 514/399; 514/415; 514/381; 514/411; 514/371; 514/367; 514/375; 514/263.4; 514/656; 514/408; 514/569; 514/438; 514/365; 514/342; 514/254.02; 514/382; 514/426; 514/352; 514/329; 514/691

(57) ABSTRACT

Ischemia-reperfusion injury remains a primary cause of morbidity and mortality in individuals who experience disruption of normal blood flow to one or more major organs. For example, there are no clinically proven strategies that prevent acute renal failure following cardiac surgery. The present invention provides a variety of methods for the treatment or prevention of ischemia-reperfusion injury. In one aspect of the invention, a method for treating or preventing ischemia-reperfusion injury includes administering to a subject an effective amount of a sphingosine kinase inhibitor. Sphingosine kinase inhibitors are very effective in the protection against IR-induced acute renal failure and liver failure. Moreover, the effects occur very early after administration, requiring only a very short time of treatment. Toxicology studies with sphingosine kinase inhibitors demonstrate that they have low toxicity, even in long-term treatment.

### CROSS-REFERENCE TO RELATED APPLICATIONS

The present application claims priority to U.S. Provisional Patent Application No. 61/176,636, filed May 8, 2009, and U.S. Provisional Patent Application No. 61/229,272 filed Jul. 28, 2009, each of which is hereby incorporated herein by reference in its entirety.

### FIELD OF THE INVENTION

The present invention relates generally to ischemia-reperfusion injury. The present invention relates more particularly to methods for preventing or ameliorating tissue damage that occurs during ischemia-reperfusion conditions.

### BACKGROUND

Ischemia-reperfusion (IR) injury refers to tissue damage that occurs following the establishment of blood flow to tissues that were previously under-perfused. For example, transplantation surgery involves the temporary cessation of blood flow to the target tissue which is followed by reestablishment of circulation upon grafting into the recipient. Less dramatic, but still clinically relevant, IR events occur during progressive diseases that result in impaired blood flow, as well as in vessel occlusions resulting from stroke or injury. A variety of biochemical mediators are involved in IR injury, including oxygen and other free radicals, ions and neurotransmitters, and inflammatory cytokines. The latter mediators exert their damaging effects, at least in part, by stimulating pathways that promote the infiltration and activation of leukocytes into the tissue resulting in irreversible damage to the tissue.

Acute renal failure (ARF) is one of the most common and serious complications following cardiac surgery (Rosner et al., J Intensive Care Med 23: 3 (2008)). The incidence of ARF is estimated to be 4-8% of all patients undergoing these procedures, with well over 450,000 procedures being performed in the United States alone each year. Mortality rates still remain around 20% for ARF patients following cardiac surgery, with survivors needing extended stays in the intensive care unit and dialysis. There are currently no effective drugs approved by the FDA for the indication of ARF prevention following cardiac surgery.

ARF frequently derives from IR injury to the kidney that occurs in cardiac surgery, elective aortic aneurysm repair, trauma, hemorrhagic and cardiac shock and kidney transplant. A variety of pathophysiological processes likely contribute to development of IR injury. Reactive oxygen species (ROS) play critical roles in the injury caused by IR. ROS not only directly damage cell membranes, DNA and protein, they also activate NF-κB, triggering the formation of toxic cytokines and chemokines (e.g. TNFα, IL-1 and MIP-2), vasoactive mediators (e.g. prostaglandins), and adhesion molecules. Ultimately this leads to local and systemic inflammatory responses, microcirculatory disturbances, tissue damage and organ failure.

IR injury to the liver occurs in hepatic surgery, particularly in liver transplantation, resection and trauma (Montalvo-Jave et al., J Surgical Res 147: 153 (2008)). The mechanism of liver IR injury involves excessive activation of inflammatory cytokines, including TNFα, IL-1β and IL-6, along with increased reactive oxygen and nitrogen species and calcium mobilization. (Shirasugi et al., Transplantation 64: 1398 (1997); Shito et al., Transplantation 63: 143 (1997)).

IR injury to the heart occurs in myocardial infarction and cardiac surgery, including transplantation. IR damage is strongly associated with elevated TNFα levels (Kon et al., Eur j Cardio-Thoracic Surg 33:215 (2008)), as well as increases in IL-1β, IL-6 and other inflammatory mediators (Moro et al., Amer J Physiol-Heart & Circ Physiol 293: H3014 (2007)). Neutralization of TNFα has been shown to attenuate damage following coronary microembolization (Skyschally et al., Circ Res 100:140 (2007)).

IR injury to the brain occurs in periods of circulatory insufficiency as well as in trauma to the head. Consequently, Traumatic Brain Injury (TBI) is the leading cause of morbidity and mortality in individuals under the age of 45 years in the world (Werner and Engelhard, Brit J Anaesthesia, 99: 4, 2007). Subsequent to direct tissue damage, impaired cerebral blood flow and metabolism lead to inflammatory processes that promote edema and excessive release of neurotransmitter, ultimately culminating in irreversible neuronal damage. Specifically, proinflammatory mediators including TNFα, IL-1β and IL-6 are upregulated within hours of injury.

There remains a need for methods for preventing or ameliorating tissue damage that occurs during ischemia-reperfusion conditions.

### BRIEF DESCRIPTION OF THE FIGURES

FIG. 1 demonstrates that ABC294640 protects against kidney damage following mild ischemia-reperfusion insult. Renal failure is indicated by elevated blood urea nitrogen (BUN) levels. Animals that were treated with ABC294640 had significantly lower BUN levels than did untreated control animals.

FIG. 2 demonstrates that ABC294640 protects against kidney damage following mild ischemia-reperfusion insult. Renal failure is indicated by elevated BUN and creatinine levels. Animals that were treated with ABC294640 had significantly lower BUN and creatinine levels than did untreated control animals.

FIG. 3 demonstrates that ABC294640 protects against death following severe ischemia-reperfusion insult. Kidney IR was performed by ligating and removing the right kidney and then clamping the left kidney for 45 minutes. All animals receiving only the vehicle treatment died within 2 days following surgery (squares). In contrast, all of the mice treated with ABC294640 survived for at least 9 days (triangles), at which time they were in good health when sacrificed. * Indicates p<0.01.

FIG. 4 demonstrates that ABC294640 protects against kidney damage following severe ischemia-reperfusion. Kidney IR was performed by ligating and removing the right kidney and then clamping the left kidney pedicle for 45 minutes. Animals receiving only the vehicle had elevated serum creatinine and BUN levels compared to those of Sham-operated animals. These levels were significantly reduced and returned to normal when treated with ABC294640 at 2 days and 10 days, respectively. ^{##} Indicates p<0.01 vs. Sham. * Indicates p<0.05 vs. Vehicle, ** Indicates p<0.01 vs. Vehicle and *** Indicates p<0.001 vs. Vehicle.

FIG. 5 demonstrates that ABC294640 reduces neutrophil infiltration into the kidney following IR. MPO activity was measured from the kidneys of animals described in FIG. 4. *** indicates p<0.001.

FIG. 6 demonstrates that ABC294640 protects against microscopic damage in the kidney following severe ischemia-reperfusion. Representative kidney sections from the animals described in FIG. 4 are shown. Panels A and C are vehicle treated animals at 4 and 48 hours, respectively. Panels B and D are ABC294640 treated animals at 4 and 48 hours, respectively. Exemplary morphological characteristics are labeled where they occur on the slides.

FIG. 7 confirms that ABC294640 protects against microscopic damage in the kidney following severe ischemia-reperfusion. Histology scores from the animals described in FIG. 4 were determined.

FIG. 8 demonstrates a correlation between kidney histology scores and serum creatinine values for the mice with ischemia-reperfusion injury. The individual histology score and serum creatinine levels in animals described in FIG. 4 are graphed. A correlation coefficient of 0.7556 was obtained, indicating a high statistical significance of p<0.01.

FIG. 9 demonstrates that ABC294640 prevents upregulation of sphingosine kinase (SK) after hepatic IR. Livers were harvested following sham-operation (sham) or 1 h-ischemia plus 6 h-reperfusion (IR). SK was detected immunohistochemcally.

FIG. 10 demonstrates that ABC294640 attenuates necrosis after hepatic IR. Livers were harvested following sham-operation (sham) or 1 h-ischemia plus 6 h-reperfusion (IR). Liver slices were stained with H+E.

FIG. 11 demonstrates that ABC294640 prevents cell death after hepatic IR. Livers were harvested following sham-operation (sham) or 1 h-ischemia plus 6 h-reperfusion (IR). Necrotic area was quantified by image analysis and apoptosis was detected by TUNEL staining. Values are mean±SEM (n=4 per group). a, p<0.05 vs sham; b, p<0.05 vs IR.

FIG. 12 demonstrates that ABC294640 improves liver function and survival after hepatic IR. Blood was collected following 1 h-ischemia plus 6 h-reperfusion (IR) for serum alanine aminotransferase (ALT) (A) and total bilirubin (B) detection. Values are mean±SEM (n=4 per group). a, p<0.05 vs sham; b, p<0.05 vs IR. Mice were observed 7 days for survival (C). Survival rates were significantly different between two groups by the Fisher Exact test.

FIG. 13 demonstrates that ABC294640 prevents mitochondrial depolarization caused by hepatic IR. After 1 h of ischemia and 2 h of reperfusion, intravital multiphoton microscopy of green Rh123 and red PI fluorescence was performed using a Zeiss LSM 510 NLO confocal/multiphoton microscope. Numbers of cells with mitochondrial depolarization were counted in 10 random fields (lower right). Values are mean±SEM. a, p<0.05 vs sham; b, p<0.05 vs IR

FIG. 14 depicts the onset of the MPT after hepatic IR. After 1 h of hepatic IR and 2 h of reperfusion in mice, intravital multiphoton microscopy of calcein-AM fluorescence was performed using a Zeiss LSM 510 NLO confocal/multiphoton microscope. (Zhong et al., Am Physiol, 295:G823-32, 2008).

FIG. 15 demonstrates that ABC294640 blunts TNFα formation and NF-κB activation after hepatic IR. Livers were harvested 2 h after IR, and TNFα mRNA was detected by real-time PCR (upper panel). Phosphorylated p65 subunit of NF-κB and actin were detected by immunoblotting (lower panel). Values are mean±SEM (n=4 per group). a, p<0.05 vs sham; b, p<0.05 vs IR.

FIG. 16 demonstrates that ABC294640 inhibits PMN infiltration after hepatic IR. Livers were harvested 6 h or 2 weeks after IR, and MPO was detected by immunohistochemical staining. Values are mean±SEM (n=3-4 per group). a, p<0.05 vs sham; b, p<0.05 vs IR.

FIG. 17 depicts the upregulation of SK after transplantation of lean and fatty livers. Liver slides were stained by Oil-Red-O staining for fatty infiltration at 20 h after saline or ethanol treatment (A). Red stained areas are shown by dotted outline. At 8 h after sham-operation (sham) or liver transplantation (Tx), liver grafts were collected and SK was detected immunohistochemically (B).

FIG. 18 demonstrates that ABC294640 decreases ALT release after LT. Blood was collected 6 h after LT for ALT measurement

FIG. 19 demonstrates that ABC294640 attenuates necrosis after transplantation of livers from non-heart-beating donors. Livers were retrieved from heart-beating (HB) or non-heart-beating (NHB) donors and transplanted (Tx). Liver grafts were harvested 18 h after implantation, and liver slices were stained with H+E.

### DESCRIPTION OF THE PREFERRED EMBODIMENTS

The present invention generally relates to methods for preventing or ameliorating tissue damage that occurs during ischemia-reperfusion conditions (e.g., involving cytokine, growth factor and chemotactic cascades, which arise during these inflammatory conditions). More particularly, one aspect of the invention is related to the use of a sphingosine kinase inhibitor as a therapeutic and/or protective agent in conditions characterized by tissue ischemia-reperfusion such as cardiac bypass surgery or other cardiac surgeries in which systemic blood flow is compromised, aortic aneurism repair, transplant surgery, other major surgical procedures, hemorrhagic shock, traumatic tissue injury, including traumatic brain injury, and/or severe hypovolemia, sepsis and hypotension. In other aspects, the invention also relates to methods for improving post-ischemic organ function in mammalian species by administering sphingosine kinase inhibitors.

In other aspects, the present invention further relates to methods for treating organ ischemia-reperfusion injury with a sphingosine kinase inhibitor alone or in combination with other therapies which prevent, ameliorate, or treat such injury. For example, in one aspect the present invention also relates to methods of treating ischemia-reperfusion injury with multiple inhibitors to cytokine/growth factors such as TNFα and IL-1β, as well as pharmaceutical compositions containing relevant cytokine or growth factor inhibitors and/or ischemia-reperfusion injury therapies. In another aspect of the invention, a sphingosine kinase inhibitor can be combined with anti-rejection drugs for the preservation of viability and function of transplanted organs in recipients.

The present invention provides methods for the use of compounds and pharmaceutical compositions for the prevention and/or treatment of ischemia-reperfusion injury. The chemical compounds and pharmaceutical compositions of the present invention may be useful, for example, in the therapy of ischemia-reperfusion injury that occurs following disruption of blood flow to the major organs. Accordingly, one aspect of the invention is a method for preventing or treating ischemia-reperfusion injury comprising delivering to a mammal a sphingosine kinase inhibitor or pharmaceutical composition containing a sphingosine kinase inhibitor.

The above-described medical problems are mediated by a common mechanism, i.e. excessive production and activity of inflammatory cytokines, providing opportunities for broad activity of targeted therapeutics. As described in more detail in the present disclosure, one such opportunity involves manipulation of sphingolipid metabolism. Sphingolipids are a major component of eukaryotic membranes. In addition, their metabolites are regulators of cellular signaling that determine the fate of cells. Inflammatory cytokines (e.g. TNFα and IL-1β and growth factors activate sphingomyelinases that hydrolyze sphingomyelin to form ceramide. Ceramidase deacylates ceramide, yielding sphingosine. Sphingosine kinase (SK) is the enzyme responsible for phosphorylation of sphingosine, forming spingosine-1-phosphate (S1P). A variety of proliferative factors and cytokines rapidly elevate cellular SK activity. Ceramide and S1P are second messengers that play important roles in the regulation of a variety of cell processes. In some cell types (e.g. myocytes, vascular smooth muscular cells, and endothelial cells), ceramide inhibits proliferation, whereas S1P stimulates cell growth and suppresses apoptosis. It is hypothesized that the relative amounts of ceramide and S1P determine the fate of cells. Since SK is the only known enzyme that phosphorylates ceramide-derived sphingosine, SK directly regulates the equilibrium of ceramide, sphingosine, and S1P.

Many studies have shown that SK regulates inflammatory cell activation. Platelets, macrophages and monocytes secrete cytokines, growth factors and S1P upon activation. Extracellular S1P activates S1P receptors, promoting inflammatory cascades at the site of tissue damage. Indeed, previous studies have shown that platelets contribute to IR injury of the transplanted organs and platelet transfusion is an independent risk factor for reduced graft survival. S1P functions as a second messenger, regulating Ca²⁺ homeostasis, cell proliferation and apoptosis. In addition, S1P induces nuclear factor kappa B (NF-κB), which in turn can increase the proinflammatory enzymes nitric oxide synthase (NOS), other cytokines and cyclooxygenase-2 (COX-2) which plays a role in inflammation through its production of prostaglandins. Oxidative and nitrative stress mediated by NOS exacerbate inflammation. Inflammatory cytokines induce adhesion molecule expression which is mediated by activation of SK and NF-κB. S1P is also a mediator of Ca²⁺ influx during granulocyte activation, leading to the production of ROS. S1P also protects granulocytes from apoptosis, which may enhance inflammation. Together, these studies indicate that activation of SK alters sphingolipid metabolism in favor of S1P formation, resulting in pro-inflammatory responses.

Altered sphingolipid metabolism has been associated with hypoxic or ischemic injury in pre-clinical models. For example, plasma S1P levels increase during myocardial infarction (Deutschman et al. Amer Heart J 146: 62 (2003)), and intracisternal delivery of a cell-permeable ceramide significantly reduces focal cerebral ischemia in hypertensive rats (Furuya et al. J Cereb Blood Flow Metab 21: 226 (2001)). In an analogous fashion, trimethylsphingosine serves a protective role for myocardium after IR injury (Muohara et al. Amer J Physiol 269: H504 (2001)). Plasma creatinine levels following renal IR were significantly lower in S1P3^{-/-} mice (Jo, et al. Kidney Int 73: 1220 (2008)). Similarly, pulmonary permeability and injury are reduced in S1P3^{-/-} mice (Gon et al. Proc Natl Acad Sci USA 102:9270 (2005)). By contrast, other studies suggest that adenoviral gene transfer of SK protects the heart against IR Injury (Duan et al. Human Gene Therap 18: 1119 (2007)). Treatment of ischemic hearts at reperfusion with S1P improved recovery of left ventricular developed pressure (Vessey et al. Med Sci Monit 12: BR318 (2006)). Therefore, the roles of SK may be organ specific, perhaps relating to the subtypes of S1P receptors.

In one embodiment of the methods of the present invention, SK in target cells or tissues in an animal undergoing reperfusion is inhibited by administering to the animal a sphingosine kinase inhibitor or a pharmaceutical composition thereof in an amount effective to inhibit SK in the target cells or tissues of the animal.

In a particularly preferred embodiment of the use of the methods of the present invention, the compounds or compositions can be used for preventing or treating organ failure in a patient requiring such treatment, by administering the compound or composition to the patient in an amount effective to inhibit the activation of target cells of said patient. For example, these methods can be used for treating a patient undergoing major surgery to protect against subsequent ischemia-reperfusion injury. This method would involve administering to the patient a compound or composition in an amount effective to inhibit SK activity in cells of the target organ.

In another particularly preferred embodiment of the use of the methods of the present invention, the compounds or compositions can be used in a method for preventing organ failure after transplantation, by administering the composition to a patient in an amount effective to inhibit the aberrant activation of SK in the transplanted organ.

In view of the beneficial effect of inhibiting SK, it is anticipated that the methods of the present invention will be useful not only for therapeutic treatment following the onset of disease, but also for the prevention of disease in animals, including humans. The methods described herein will be essentially the same whether the compounds or pharmaceutical compositions are being administered for the treatment or prevention of disease.

In one embodiment of the invention, the ischemia-reperfusion injury is due to a surgical procedure, such as, for example, cardiac bypass surgery, aortic aneurysm repair, or organ transplant.

In another embodiment of the invention, the ischemia-reperfusion injury is due to hemorrhagic shock.

In another embodiment of the invention, the ischemia-reperfusion injury is due to trauma.

In another embodiment of the invention, the ischemia-reperfusion injury is due to a stroke resulting from cerebral infarction, cerebral hemorrhage, subarachnoid hemorrhage, or transient cerebral ischemia.

In another embodiment of the invention, the ischemia-reperfusion injury is due to a myocardial infarction.

In another embodiment of the invention, the ischemia-reperfusion injury is due to sepsis.

In another embodiment of the invention, the ischemia-reperfusion injury is due to hypotension.

In various embodiments, the ischemia-reperfusion injury occurs in the kidney; the brain; the heart; or the liver. However, in certain embodiments, the ischemia-reperfusion injury does not occur in the liver.

In spite of the high interest in sphingolipid-related signaling, there are few known inhibitors of SK. The present inventors and their coworkers have identified a series of structurally novel inhibitors of SK (French et al. Cancer Res 63(18): 5962 (2003); French et al. J Pharmacol Exp Ther 318(2): 596 (2006); Maines et al. Digest Dis Sci 53(4): 997 (2008); Maines et al. Invest Ophthalmol V is Sci 47(11): 5022 (2006)). They inhibit both recombinant human SK and endogenous S1P formation in intact cells (French et al. Cancer Res 63(18): 5962 (2003)). These SK inhibitors have activity in cell and animal models, inhibiting ulcerative colitis and cancer in the absence of systemic toxicity (French et al. J Pharmacol Exp Ther 318(2): 596 (2006); Maines et al. Digest Dis Sci 53(4): 997 (2008); Maines et al. Invest Ophthalmol V is Sci 47(11): 5022 (2006)). Each of the above-referenced publications describes sphingosine kinase inhibitors suitable for use in certain embodiments of the invention, and is hereby incorporated by reference in its entirety. Inhibitors of sphingosine kinase and prodrugs thereof useful in certain embodiments of the present invention are also described in U.S. Pat. No. 7,338,961, U.S. Patent Application Publications nos. 2006/0287317 and 2007/0032531, and International Patent Application no. US2010/027177, each of which is hereby incorporated by reference in its entirety.

For example, in one embodiment of the invention, the sphingosine kinase inhibitor is 3-(4-chlorophenyl)-N-(pyridin-4-ylmethyl)adamantane-1-carboxamide (ABC294640) or a pharmaceutically acceptable salt thereof. In another embodiment of the invention, the sphingosine kinase inhibitor is 3-(4-chlorophenyl)-N-(2-(3,4-dihydroxyphenyl)ethyl)adamantane-1-carboxamide or a pharmaceutically acceptable salt thereof.

In another embodiment of the invention, the sphingosine kinase inhibitor is safingol (dihydrosphingosine), N,N-dimethylsphingosine, or (as described by French et al. Cancer Res. 63(18): 5962 (2003)) 5-naphthalen-2-yl-2H-pyrazole-3-carboxylic acid (2-hydroxy-naphthalen-1-ylmethylene)-hydrazide (Compound I, CAS#306301-68-8); 2-(p-hydroxyanilino)-4-(p-chlorophenyl)thiazole (Compound II, CAS#312636-16-1); 5-(2,4-dihydroxy-benzylidene)-3-(4-methoxy-phenyl)-2-thioxo-thiazolidin-4-one (Compound III, CAS#359899-55-1); 2-(3,4-dihydroxy-benzylidene)-benzo[b]thiophen-3-one (Compound IV, CAS#24388-08-7); 2-(3,4-dihydroxy-benzylidene)-benzofuran-3-one (Compound V), B-5354a, b, or c (Kono et al. J Antibiotics 53: 753 (2000)), F-12509A (Kono et al. J Antibiotics 53(5): 459 (2000)), or S-15183a or b (Kono et al. J Antibiotics 54: 415 (2001)). Each of the above-described references is hereby incorporated herein by reference in its entirety.

In other embodiments the sphingosine kinase inhibitor is a compound having structural formula (I):

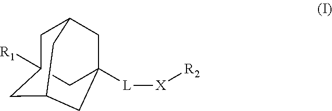

or a pharmaceutically acceptable salts thereof, wherein

L is a bond or is —C(R₃,R₄)—;

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, −S(O)₂N(R₄)— or —N(R₄)S(O)₂—;

R₁ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, mono or dialkylthiocarbamoyl, alkyl-S-alkyl, -heteroaryl-aryl, -alkyl-heteroaryl-aryl, —C(O)—NH-aryl, -alkenyl-heteroaryl, —C(O)-heteroaryl, or -alkenyl-heteroaryl-aryl;

R₃ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above R₁, R₂, and R₃ groups is optionally substituted with up to 5 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or NR′R″, wherein R′ and R″ are independently H or (C₁-C₆) alkyl, and wherein each alkyl portion of a substituent is optionally further substituted with 1, 2, or 3 groups independently selected from halogen, CN, OH, NH₂; and

R₄ and R₅ are independently H or alkyl, provided that when R₃ and R₄ are on the same carbon, and R₃ is oxo, then R₄ is absent.

In certain embodiments of the compounds of structural formula (I) as described above, L is a bond.

In certain embodiments of the compounds of structural formula (I) as described above, L is a bond and X is —C(R₃R₄)—. For example, X can be —C(O)—.

In certain embodiments of the compounds of structural formula (I) as described above, R₁ is H.

In certain embodiments of the compounds of structural formula (I) as described above, R₁ is optionally substituted aryl, for example, phenyl. In certain embodiments, the phenyl is unsubstituted. In other embodiments, the phenyl is substituted with halogen (e.g., monohalo-substituted at the 4-position. Preferred halogen substituents are Cl and F.

In certain embodiments of the compounds of structural formula (I) as described above, R₂ is OH.

In certain embodiments of the compounds of structural formula (I) as described above, R₂ is C₁-C₆ alkyl, for example, C₁-C₃ alkyl (e.g., CH₃).

In certain embodiments of the compounds of structural formula (I) as described above, R₂ is alkenylaryl. Preferably, the aryl portion of alkenylaryl is phenyl or naphthyl, optionally substituted with 1 or 2 of halogen, cyano, or hydroxy.

In certain embodiments of the compounds of structural formula (I) as described above, R₂ is -alkenyl-heteroaryl.

In certain embodiments of the compounds of structural formula (I) as described above, R₂ is -alkenyl-heteroaryl-aryl.

Certain preferred compounds of structural formula (I) as described above include compounds of structural formula (I-1):

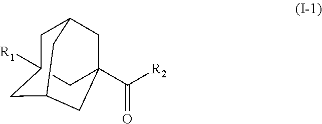

and pharmaceutically acceptable salts thereof, wherein:

R₁ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl; and

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, mono or dialkylthiocarbamoyl, alkyl-S-alkyl, -heteroaryl-aryl, -alkyl-heteroaryl-aryl, —NH-aryl, -alkenyl-heteroaryl, -heteroaryl, —NH-alkyl, —NH-cycloalkyl, or -alkenyl-heteroaryl-aryl,

wherein the alkyl and ring portion of each of the above R₁, and R₂ groups is optionally substituted with up to 5 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or NR′R″, wherein R′ and R″ are independently H or (C₁-C₆) alkyl, and wherein each alkyl portion of a substituent is optionally further substituted with 1, 2, or 3 groups independently selected from halogen, CN, OH, NH₂.

Certain preferred compounds of structural formula (I) as described above include those of structural formula (II):

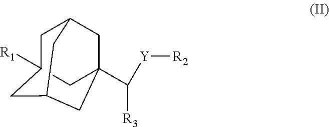

and pharmaceutically acceptable salts thereof, wherein:

Y is —C(R₄,R₅)—, —N(R₄)—, —O—, or —C(O)—;

R₁ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, mono or dialkylthiocarbamoyl, alkyl-S-alkyl, -heteroaryl-aryl, -alkyl-heteroaryl-aryl, —C(O)—NH-aryl, -alkenyl-heteroaryl, —C(O)-heteroaryl, or -alkenyl-heteroaryl-aryl;

R₃ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocaxbamoyl;

wherein the alkyl and ring portion of each of the above R₁, R₂, and R₃ groups is optionally substituted with up to 5 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or NR′R″, wherein R′ and R″ are independently H or (C₁-C₆) alkyl, and wherein each alkyl portion of a substituent is optionally further substituted with 1, 2, or 3 groups independently selected from halogen, CN, OH, NH₂; and

R₄ and R₅ are independently H or alkyl.

In certain embodiments of the compounds of structural formula (II) as described above,

Y is —C(R₄,R₅)— or —N(R₄)—;

R₁ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, mono or dialkylthiocarbamoyl, alkyl-S-alkyl, -heteroaryl-aryl, -alkyl-heteroaryl-aryl, —C(O)—NH-aryl, -alkenyl-heteroaryl, —C(O)-heteroaryl, or -alkenyl-heteroaryl-aryl;

wherein the alkyl and ring portion of each of the above R₁ and R₂ groups is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅, and wherein each alkyl portion of a substituent is optionally further substituted with 1, 2, or 3 groups independently selected from halogen, CN, OH, NH₂;

R₃ is H, alkyl, or oxo (═O); and

R₄ and R₅ are independently H or (C₁-C₆)alkyl. In certain embodiments of the compounds of structural formula (II) as described above, Y is —NH—.

In certain embodiments of the compounds of structural formula (II) as described above, X is —C(O)—.

In certain embodiments of the compounds of structural formula (II) as described above, R₃ is methyl.

In certain embodiments of the compounds of structural formula (II) as described above, R₁ is H.

In certain embodiments of the compounds of structural formula (II) as described above, R₁ is optionally substituted aryl. Preferably, the aryl is phenyl, either unsubstituted or substituted with 1 or 2 halogen groups. Preferably, halogen is chloro or fluoro.

In certain embodiments of the compounds of structural formula (II) as described above, R₂ is alkyl or cycloalkyl.

In certain embodiments of the compounds of structural formula (II) as described above, R₂ is aryl or -alkylaryl (e.g., phenyl or -alkyl-phenyl). The -alkyl- can be, for example, C₁-C₃-alkyl-, either straight chain or branched. The aryl groups may be unsubstituted or substituted. In certain embodiments, the substituents include 1, 2, 3, 4, or 5 (e.g., 1 or 2) groups independently chosen from halogen, hydroxy, alkyl, cyanoalkyl, aminoalkyl, thioalkoxy, trifluoromethyl, haloalkoxy, aryloxy, and alkoxy.

In certain embodiments of the compounds of structural formula (II) as described above, R₂ is heterocycloalkyl or -alkyl-heterocycloalkyl. The -alkyl- can be, for example, C₁-C₃-alkyl-, either straight chain or branched. The heterocycloalkyl in either group may be, for example, piperidinyl, piperazinyl, pyrrolidinyl, and morpholinyl. The heterocycloalkyl groups may be unsubstituted or substituted. Preferred substituents include 1, 2, 3, 4, or 5 (preferably 1 or 2) groups independently chosen from halogen, hydroxy, alkyl, cyanoalkyl, aminoalkyl, thioalkoxy, trifluoromethyl, haloalkoxy, aryloxy, oxo, and alkoxy.

In certain embodiments of the compounds of structural formula (II) as described above, R₂ is heteroaryl or -alkyl-heteroaryl. The -alkyl- can be, for example, C₁-C₃-alkyl-, either straight chain or branched. The heteroaryl in either group may be, for example, pyridinyl, imidazolyl, indolyl, carbazolyl, thiazolyl, benzothiazolyl, benzooxazolyl, purinyl, and thienyl. The heteroaryl groups may be unsubstituted or substituted. Preferred substituents include 1, 2, 3, 4, or 5 (preferably 1 or 2) groups independently chosen from halogen, hydroxy, alkyl, cyanoalkyl, aminoalkyl, thioalkoxy, trifluoromethyl, haloalkoxy, aryloxy, oxo, and alkoxy.

Compounds according to structural formula (I), (I-1) and (II) are described in U.S. Patent Application Publication no. 2006/0287317, which is hereby incorporated herein by reference in its entirety. Specific example compounds are described in more detail therein, and in the Examples below.

In other embodiments the sphingosine kinase inhibitor is a compound having structural formula (III):

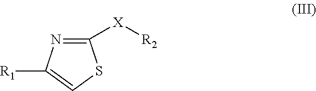

or a pharmaceutically acceptable salt thereof, wherein

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, −S(O)₂N(R₄)— or —N(R₄)S(O)₂—;

R₁ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above R₁ and R₂ groups is optionally substituted with up to 5 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or NR′R″, wherein R′ and R″ are independently H or (C₁-C₆) alkyl, and wherein each alkyl portion of a substituent is optionally further substituted with 1, 2, or 3 groups independently selected from halogen, CN, OH, NH₂;

R₃ is H, alkyl, preferably lower alkyl, or oxo, provided that when R₃ and R₄ are on the same carbon, and R₃ is oxo, then R₄ is absent; and

R₄ and R₅ are independently H or alkyl, preferably lower alkyl.

In other embodiments the sphingosine kinase inhibitor is a compound having structural formula (IV):

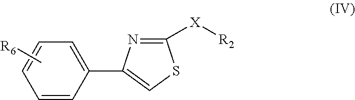

or a pharmaceutically acceptable salt thereof, wherein:

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅;

R₃ is H, alkyl, preferably lower alkyl, or oxo, provided that when R₃ and R₄ are on the same carbon, and R₃ is oxo, then R₄ is absent;

R₄ and R₅ are independently H or (C₁-C₆)alkyl; and

R₆ is halogen, haloalkyl, alkoxy, haloalkoxy, hydroxyalkyl, alkanoyl, —COOH, —OH, —SH, —S-alkyl, —CN, —NO₂, or —NH₂.

In certain embodiments of the compounds of structural formula (IV) as described above,

X is —C(R₃,R₄)N(R₅)—, —C(O)N(R₄)—, —N(R₄)C(O)—, or —C(R₄,R₅)—;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent;

R₄ and R₅ are independently H or (C₁-C₆)alkyl; and

R₆ is halogen, haloalkyl, alkoxy, haloalkoxy, hydroxyalkyl, alkanoyl, —COOH, —OH, —SH, —S-alkyl, —CN, —NO₂, or —NH₂.

In certain embodiments of the compounds of structural formula (IV) as described above:

X is —C(O)N(R₄)— or —N(R₄)C(O)—;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₄ and R₅ are independently H or (C₁-C₆)alkyl and

R₆ is halogen, haloalkyl, alkoxy, haloalkoxy, hydroxyalkyl, alkanoyl, —COOH, —OH, —SH, —S-alkyl, —CN, —NO₂, or —NH₂.

In other embodiments the sphingosine kinase inhibitor is a compound having structural formula (V):

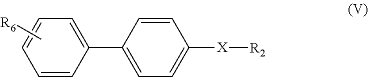

or a pharmaceutically acceptable salt thereof, wherein:

X is —C(R₃,R₄)N(R₅)—, —C(O)N(R₄)—, —N(R₄)C(O)—, —C(R₄,R₅)—, —N(R₄)—, —O—, —S—, —C(O)—, —S(O)₂—, —S(O)₂N(R₄)— or —N(R₄)S(O)₂—;

R₁ is halogen, haloalkyl, alkoxy, haloalkoxy, hydroxyalkyl, alkanoyl, —COOH, —OH, —SH, —S-alkyl, —CN, —NO₂, or —NH₂:

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent; and

R₄ and R₅ are independently H or (C₁-C₆)alkyl.

In certain embodiments of the compounds of structural formula (IV) as described above,

X is —C(R₃,R₄)N(R₅)—, —C(O)N(R₄)—, —N(R₄)C(O)—, or —C(R₄,R₅)—;

R₁ is halogen, haloalkyl, alkoxy, haloalkoxy, hydroxyalkyl, alkanoyl, —COOH, —OH, —SH, —S-alkyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent; and

R₄ and R₅ are independently H or (C₁-C₆)alkyl. In certain embodiments of the compounds of structural formula (V) as described above:

X is —C(O)N(R₄)— or —N(R₄)C(O)—;

R₁ is halogen, haloalkyl, alkoxy, haloalkoxy, hydroxyalkyl, alkanoyl, —COOH, —OH, —SH, —S-alkyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent; and

R₄ and R₅ are independently H or (C₁-C₆)alkyl. In other embodiments the sphingosine kinase inhibitor is a compound having structural formula (VI):

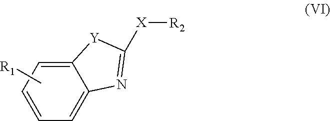

or a pharmaceutically acceptable salt thereof, wherein:

X is —C(R₃,R₄)N(R₅)—, —C(O)N(R₄)—, —N(R₄)C(O)—, —C(R₄,R₅)—, —O—, —S—, —C(O)—, —S(O)₂—, —S(O)₂N(R₄)— or —N(R₄)S(O)₂—;

Y is O or S;

R₁ is halogen, haloalkyl, alkoxy, haloalkoxy, hydroxyalkyl, alkanoyl, —COOH, —OH, —SH, —S-alkyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent; and

R₄ and R₅ are independently H or (C₁-C₆)alkyl. In certain embodiments of the compounds of structural formula (VI) as described above,

X is —C(R₃,R₄)N(R₅)—, —C(O)N(R₄)—, —N(R₄)C(O)—, or —C(R₄,R₅)—;

Y is O or S;

R₁ is halogen, haloalkyl, alkoxy, haloalkoxy, hydroxyalkyl, alkanoyl, —COOH, —OH, —SH, —S-alkyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃ is H, alkyl, preferably lower alkyl, or oxo, provided that when R₃ and R₄ are on the same carbon, and R₃ is oxo, then R₄ is absent; and

R₄ and R₅ are independently H or (C₁-C₆)alkyl.

In certain embodiments of the compounds of structural formula (VI) as described above,

X is —C(O)N(R₄)— or —N(R₄)C(O)—;

Y is O or S;

R₁ is halogen, haloalkyl, alkoxy, haloalkoxy, hydroxyalkyl, alkanoyl, —COOH, —OH, —SH, —S-alkyl, —CN, —NO₂, or —NH₂;

R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;

wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and

R₃, R₄ and R₅ are independently H or (C₁-C₆)alkyl.

In certain embodiments of the compounds of structural formulae (III)-(VI) as described above, X is —C(O)—.

In certain embodiments of the compounds of structural formulae (III)-(VI) as described above, R₃ is methyl.

In certain embodiments of the compounds of structural formulae (III)-(VI) as described above, R₁ is H.

In certain embodiments of the compounds of structural formulae (III)-(VI) as described above, R₁ is optionally substituted aryl. Preferably, the aryl is phenyl, either unsubstituted or substituted with 1 or 2 halogen groups. Preferably, halogen is chloro or fluoro.

In certain embodiments of the compounds of structural formulae (III)-(VI) as described above, R₂ is alkyl or cycloalkyl.

In certain embodiments of the compounds of structural formulae (III)-(VI) as described above, R₂ is aryl or -alkylaryl (e.g., phenyl or -alkyl-phenyl). The -alkyl- can be, for example, C₁-C₃-alkyl-, either straight chain or branched. The aryl groups may be unsubstituted or substituted. In certain embodiments, the substituents include 1, 2, 3, 4, or 5 (e.g., 1 or 2) groups independently chosen from halogen, hydroxy, alkyl, cyanoalkyl, aminoalkyl, thioalkoxy, trifluoromethyl, haloalkoxy, aryloxy, and alkoxy.

In certain embodiments of the compounds of structural formulae (III)-(VI) as described above, R₂ is heterocycloalkyl or -alkyl-heterocycloalkyl. The -alkyl- can be, for example, C₁-C₃-alkyl-, either straight chain or branched. The heterocycloalkyl in either group may be, for example, piperidinyl, piperazinyl, pyrrolidinyl, and morpholinyl. The heterocycloalkyl groups may be unsubstituted or substituted. Preferred substituents include 1, 2, 3, 4, or 5 (preferably 1 or 2) groups independently chosen from halogen, hydroxy, alkyl, cyanoalkyl, aminoalkyl, thioalkoxy, trifluoromethyl, haloalkoxy, aryloxy, oxo, and alkoxy.

In certain embodiments of the compounds of structural formulae (III)-(VI) as described above, R₂ is heteroaryl or -alkyl-heteroaryl. The -alkyl- can be, for example, C₁-C₃-alkyl-, either straight chain or branched. The heteroaryl in either group may be, for example, pyridinyl, imidazolyl, indolyl, carbazolyl, thiazolyl, benzothiazolyl, benzooxazolyl, purinyl, and thienyl. The heteroaryl groups may be unsubstituted or substituted. Preferred substituents include 1, 2, 3, 4, or 5 (preferably 1 or 2) groups independently chosen from halogen, hydroxy, alkyl, cyanoalkyl, aminoalkyl, thioalkoxy, trifluoromethyl, haloalkoxy, aryloxy, oxo, and alkoxy.

Compounds according to structural formula (III)-(VI) are described in U.S. Patent Application Publication no. 2007/0032531, which is hereby incorporated herein by reference in its entirety. Specific example compounds are described in more detail therein, and in the Examples below.

The compounds and pharmaceutically acceptable salts described herein can be provided as pharmaceutical compositions, comprising the compound or salt as active ingredient, in combination with a pharmaceutically acceptable carrier, medium, or auxiliary agent.

The pharmaceutical compositions may be prepared in various forms for administration, including tablets, caplets, pills or dragees, or can be filled in suitable containers, such as capsules, or, in the case of suspensions, filled into bottles. As used herein “pharmaceutically acceptable carrier medium” includes any and all solvents, diluents, or other liquid vehicle; dispersion or suspension aids; surface active agents; preservatives; solid binders; lubricants and the like, as suited to the particular dosage form desired. Various vehicles and carriers used in formulating pharmaceutical compositions and known techniques for the preparation thereof are disclosed in Remington's Pharmaceutical Sciences (Osol et al. eds., 15th ed., Mack Publishing Co.: Easton, Pa., 1975). Except insofar as any conventional carrier medium is incompatible with the chemical compounds described herein, such as by producing any undesirable biological effect or otherwise interacting in a deleterious manner with any other component of the pharmaceutical composition, the use of the carrier medium is contemplated to be within the scope of this invention.

In the pharmaceutical compositions, the active agent may be present, for example, in an amount of at least 1% and not more than 99% by weight, based on the total weight of the composition, including carrier medium or auxiliary agents. Preferably, the proportion of active agent varies between 1% to 70% by weight of the composition. Pharmaceutical organic or inorganic solid or liquid carrier media suitable for enteral or parenteral administration can be used to make up the composition. Gelatin, lactose, starch, magnesium, stearate, talc, vegetable and animal fats and oils, gum polyalkylene glycol, or other known excipients or diluents for medicaments may all be suitable as carrier media.

The pharmaceutical compositions may be administered using any amount and any route of administration effective for treating a patient as described herein. Thus the expression “therapeutically effective amount,” as used herein, refers to a sufficient amount of the active agent to provide the desired effect against target cells. The exact amount required will vary from subject to subject, depending on the species, age, and general condition of the subject; the severity of the ischemia-reperfusion injury; the particular SK inhibitor; its mode of administration; and the like.

The pharmaceutical compounds are preferably formulated in unit dosage form for ease of administration and uniformity of dosage. “Unit dosage form,” as used herein, refers to a physically discrete unit of therapeutic agent appropriate for the animal to be treated. Each dosage should contain the quantity of active material calculated to produce the desired therapeutic effect either as such, or in association with the selected pharmaceutical carrier medium. Typically, the pharmaceutical composition will be administered in dosage units containing from about 0.1 mg to about 10,000 mg of the agent, with a range of about 1 mg to about 1000 mg being preferred.

The pharmaceutical compositions may be administered orally or parentally, such as by intramuscular injection, intraperitoneal injection, or intravenous infusion. The pharmaceutical compositions may be administered orally or parenterally at dosage levels of about 0.1 to about 1000 mg/kg, and preferably from about 1 to about 100 mg/kg, of animal body weight per day, one or more times a day, to obtain the desired therapeutic effect.

Although the pharmaceutical compositions can be administered to any mammal that can benefit from the therapeutic effects of the compositions, the compositions are intended particularly for the treatment of diseases in humans.

The pharmaceutical compositions will typically be administered from 1 to 4 times a day, so as to deliver the daily dosage as described herein. Alternatively, dosages within these ranges can be administered by constant infusion over an extended period of time, usually 1 to 96 hours, until the desired therapeutic benefits have been obtained. However, the exact regimen for administration of the chemical compounds and pharmaceutical compositions described herein will necessarily be dependent on the needs of the animal being treated, the type of treatments being administered, and the judgment of the attending physician.

In certain situations, the compounds described herein may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric sythesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

Non-toxic pharmaceutically acceptable salts of the compounds described herein include, but are not limited to salts of inorganic acids such as hydrochloric, sulfuric, phosphoric, diphosphoric, hydrobromic, and nitric or salts of organic acids such as formic, citric, malic, maleic, fumaric, tartaric, succinic, acetic, lactic, methanesulfonic, p-toluenesulfonic, 2-hydroxyethylsulfonic, salicylic and stearic. Similarly, pharmaceutically acceptable cations include, but are not limited to sodium, potassium, calcium, aluminum, lithium and ammonium. Those skilled in the art will recognize a wide variety of non-toxic pharmaceutically acceptable addition salts. The invention also encompasses prodrugs of the compounds described herein, such as those described in International Patent Application no. US2010/027177.

Those skilled in the art will recognize various synthetic methodologies, which may be employed to prepare the compounds described herein, as well as non-toxic pharmaceutically acceptable addition salts and prodrugs of the compounds described herein.

### DEFINITIONS

The definitions and explanations below are for the terms as used throughout this entire document, including both the specification and the claims.

It should be noted that, as used in this specification and the appended claims, the singular fauns “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

The symbol “-” in general represents a bond between two atoms in the chain. Thus CH₃—O—CH₂—CH(Rᵢ)—CH₃ represents a 2-substituted-1-methoxypropane compound. In addition, the symbol “-” represents the point of attachment of the substituent to a compound. Thus for example aryl(C₁-C₆)alkyl- indicates an alkylaryl group, such as benzyl, attached to the compound at the alkyl moiety.

Where multiple substituents are indicated as being attached to a structure, it is to be understood that the substituents can be the same or different. Thus for example “Rₘ optionally substituted with 1, 2 or 3 R_{q} groups” indicates that Rₘ is substituted with 1, 2, or 3 R_{q} groups where the R_{q} groups can be the same or different.

The phrase “optionally substituted” is used interchangeably with the phrase “substituted or unsubstituted”. Unless otherwise indicated, an optionally substituted group may have a substituent at each substitutable position of the group, and each substituent is independent of the other.

As used herein, the terms “halogen” or “halo” indicate fluorine, chlorine, bromine, or iodine.

The term “heteroatom” means nitrogen, oxygen or sulfur and includes any oxidized form of nitrogen and sulfur, and the quaternized form of any basic nitrogen. Also the term “nitrogen” includes a substitutable nitrogen in a heterocyclic ring. As an example, in a saturated or partially unsaturated ring having 0-3 heteroatoms selected from nitrogen, oxygen or sulfur, the nitrogen may be N (as in 3,4-dihydro-2H-pyrrolyl), NH (as in pyrrolidinyl) or NR⁺ (as in N-substituted pyrrolidinyl).

The term “alkyl”, as used herein alone or as part of a larger moiety, refers to a saturated aliphatic hydrocarbon including straight chain, branched chain or cyclic (also called “cycloalkyl”) groups. Examples of alkyl groups include methyl, ethyl, propyl, isopropyl, butyl, iso-, sec- and tert-butyl, pentyl, hexyl, heptyl, 3-ethylbutyl, and the like. Preferably, the alkyl group has 1 to 20 carbon atoms (whenever a numerical range, e.g. “1-20”, is stated herein, it means that the group, in this case the alkyl group, may contain 1 carbon atom, 2 carbon atoms, 3 carbon atoms, etc. up to and including 20 carbon atoms). More preferably, it is a medium size alkyl having 1 to 10 carbon atoms. Most preferably, it is a lower alkyl having 1 to 4 carbon atoms. The cycloalkyl can be monocyclic, or a polycyclic fused system. Examples of cycloalkyl groups include cyclopropyl, cyclobutyl, cyclolpentyl, cyclohexyl, cycloheptyl, cyclooctyl, and adamantyl. The alkyl or cycloalkyl group may be unsubstituted or substituted with 1, 2, 3 or more substituents. Examples of such substituents including, without limitation, halo, hydroxy, amino, alkoxy, alkylamino, dialkylamino, cycloalkly, aryl, aryloxy, arylalkyloxy, heterocyclic radical, and (heterocyclic radical)oxy. Examples include fluoromethyl, hydroxyethyl, 2,3-dihydroxyethyl, (2- or 3-furanyl)methyl, cyclopropylmethyl, benzyloxyethyl, (3-pyridinyl)methyl, (2-thienyl)ethyl, hyroxypropyl, aminocyclohexyl, 2-dimethylaminobutyl, methoxymethyl, N-pyridinylethyl, and diethylaminoethyl.

The term “cycloalkylalkyl”, as used herein alone or as part of a larger moiety, refers to a C₃-C₁₀ cycloalkyl group attached to the parent molecular moiety through an alkyl group, as defined above. Examples of cycloalkylalkyl groups include cyclopropylmethyl and cyclopentylethyl.

The term “alkenyl”, as used herein alone or as part of a larger moiety, refers to an aliphatic hydrocarbon having at least one carbon-carbon double bond, including straight chain, branched chain or cyclic groups having at least one carbon-carbon double bond. Preferably, the alkenyl group has 2 to 20 carbon atoms. More preferably, it is a medium size alkenyl having 2 to 10 carbon atoms. Most preferably, it is a lower alkenyl having 2 to 6 carbon atoms. The alkenyl group may be unsubstituted or substituted with 1, 2, 3 or more substituents. Examples of such substituents including, without limitation halo, hydroxy, amino, alkoxy, alkylamino, dialkylamino, cycloalkly, aryl, aryloxy, arylalkyloxy, heterocyclic radical, and (heterocyclic radical)oxy. Depending on the placement of the double bond and substituents, if any, the geometry of the double bond may be entgegen (E) or zusammen (Z), cis, or trans. Examples of alkenyl groups include ethenyl, propenyl, cis-2-butenyl, trans-2-butenyl, and 2-hyroxy-2-propenyl.

The term “alkynyl”, as used herein alone or as part of a larger moiety, refers to an aliphatic hydrocarbon having at least one carbon-carbon triple bond, including straight chain, branched chain or cyclic groups having at least one carbon-carbon triple bond. Preferably, the alkynyl group has 2 to 20 carbon atoms. More preferably, it is a medium size alkynyl having 2 to 10 carbon atoms. Most preferably, it is a lower alkynyl having 2 to 6 carbon atoms. The alkynyl group may be unsubstituted or substituted with 1, 2, 3 or more substituents. Examples of such substituents including, without limitation, halo, hydroxy, amino, alkoxy, alkylamino, dialkylamino, cycloalkly, aryl, aryloxy, arylalkyloxy, heterocyclic radical, and (heterocyclic radical)oxy. Examples of alkynyl groups include ethynyl, propynyl, 2-butynyl, and 2-hyroxy-3-butylnyl.

The term “alkoxy”, as used herein alone or as part of a larger moiety, represents an alkyl group of indicated number of carbon atoms attached to the parent molecular moiety through an oxygen bridge. Examples of alkoxy groups include, for example, methoxy, ethoxy, propoxy and isopropoxy. Alkoxy radicals may be further substituted with one or more halo atoms, such as fluoro, chloro or bromo, to provide “haloalkoxy” radicals. Examples of such radicals include fluoromethoxy, chloromethoxy, trifluoromethoxy, and fluoroethoxy.

The term “aryl”, as used herein alone or as part of a larger moiety, refers to an aromatic hydrocarbon ring system containing at least one aromatic ring. The aromatic ring may optionally be fused or otherwise attached to other aromatic hydrocarbon rings or non-aromatic hydrocarbon rings. Additionally, the aryl group may be substituted or unsubstituted by various groups such as hydrogen, halo, hydroxy, alkyl, haloalkyl, alkoxy, nitro, cyano, alkylamine, carboxy or alkoxycarbonyl. Examples of aryl groups include, for example, phenyl, naphthyl, 1,2,3,4-tetrahydronaphthalene, benzodioxole, and biphenyl. Preferred examples of unsubstituted aryl groups include phenyl and biphenyl. Preferred aryl group substituents include hydrogen, halo, alkyl, haloalkyl, hydroxy and alkoxy.

The term “heteroalkyl”, as used herein alone or as part of a larger moiety, refers to an alkyl radical as defined herein with one or more heteroatoms replacing a carbon atom with the moiety. Such heteroalkyl groups are alternately referred to using the terms ether, thioether, amine, and the like.

The term “heterocyclyl”, as used herein alone or as part of a larger moiety, refers to saturated, partially unsaturated and unsaturated heteroatom-containing ring shaped radicals, where the heteroatoms may be selected from nitrogen, sulfur and oxygen. Said heterocyclyl groups may be unsubstituted or substituted at one or more atoms within the ring system. The heterocyclic ring may contain one or more oxo groups.

The term “heterocycloalkyl”, as used herein alone or as part of a larger moiety, refers to a non-aromatic ring system containing at least one heteroatom selected from nitrogen, oxygen, and sulfur. The heterocycloalkyl ring may be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred monocyclic heterocycloalkyl groups include piperidyl, piperazinyl, morpholinyl, pyrrolidinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like. Heterocycloalkyl radicals may also be partially unsaturated. Examples of such groups include dihydrothienyl, dihydropyranyl, dihydrofuryl, and dihydrothiazolyl.

The term “heteroaryl”, as used herein alone or as part of a larger moiety, refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Additionally, the heteroaryl group may be unsubstituted or substituted at one or more atoms of the ring system, or may contain one or more oxo groups. Examples of heteroaryl groups include, for example, pyridine, furan, thiophene, carbazole and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, benzopyrazolyl, purinyl, benzooxazolyl, and carbazolyl.

The term “acyl” means an H—C(O)— or alkyl-C(O)— group in which the alkyl group, straight chain, branched or cyclic, is as previously described. Exemplary acyl groups include formyl, acetyl, propanoyl, 2-methylpropanoyl, butanoyl, and caproyl.

The term “aroyl” means an aryl-C(O)— group in which the aryl group is as previously described. Exemplary aroyl groups include benzoyl and 1- and 2-naphthoyl.

The term “solvate” means a physical association of a compound described herein with one or more solvent molecules. This physical association involves varying degress of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Exemplary solvates include ehanolates, methanolates, and the like. “Hydrate” is a solvate wherein the solvent molecule(s) is/are H₂O.

Compounds that have the same molecular formula but differ in the nature or sequence of bonding of their atoms or arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, a carbon atom that is bonded to four different groups, a pair of enantiomers is possible. An enantiomer can be characterized by the absolute configuration of its asymmetric center and is described by the R- and S-sequencing rules of Calm and Prelog, which are well known to those in the art. Additionally, enantiomers can be characterized by the manner in which a solution of the compound rotates a plane of polarized light and designated as dextrorotatory or levorotatory (i.e. as (+) or (−) isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless otherwise indicated, the specification and claims is intended to include both individual enantiomers as well as mixtures, racemic or otherwise, thereof.

Certain compounds described herein may exhibit the phenomena of tautomerism and/or structural isomerism. For example, certain compounds described herein may adopt an E or a Z configuration about a carbon-carbon double bond or they may be a mixture of E and Z. This invention encompasses any tautomeric or structural isomeric form and mixtures thereof.

Unless otherwise stated, structures depicted herein are also meant to include compounds that differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are within the scope of this invention. Such compounds are useful, for example, as analytical tools or probes in biologic assays.

The term “method” refers to manners, means, techniques and procedures for accomplishing a given task including, but not limited to, those manners, means, techniques and procedures either known to, or readily developed from known manners, means, techniques and procedures by practitioners of the chemical, pharmaceutical, biological, biochemical and medical arts. The methods described herein may also be practiced as uses of the compounds described herein for the preparation of medicaments for use in treating or preventing the injuries and disorders described herein.

The term “IC₅₀” or “50% inhibitory concentration” as used herein refers to the concentration of a compound that reduces a biological process by 50%. These processes can include, but are not limited to, enzymatic reactions, i.e. inhibition of SK catalytic activity, or cellular properties, i.e. cell proliferation, apoptosis or cellular production of S1P. The sphingosine kinase inhibitory activity of the compounds described herein can be determined as described in the following two paragraphs

An assay for identifying inhibitors of recombinant human SK has been established (French et al., 2003, Cancer Res 63: 5962). cDNA for human SK is subcloned into a pGEX bacterial expression vector, which results in expression of the enzyme as a fusion protein with glutathione-S-transferase, and the fusion protein is then purified on a column of immobilized glutathione. SK activity is measured by incubation of the recombinant SK with [³H]sphingosine and 1 mM ATP under defined conditions, followed by extraction of the assay mixture with chloroform:methanol under basic conditions. This results in the partitioning of the unreacted [³H]sphingosine into the organic phase, while newly synthesized [³H]S1P partitions into the aqueous phase. Radioactivity in aliquots of the aqueous phase is then quantified as a measure of [³H]S1P formation. There is a low background level of partitioning of [³H]sphingosine into the aqueous phase, and addition of the recombinant SK greatly increases the formation of [³H]S1P. A positive control, DMS, completely inhibits SK activity at concentrations above 25 μM.

In an alternate assay procedure, the recombinant human SK is incubated with unlabeled sphingosine and ATP as described above. After 30 minutes, the reactions were terminated by the addition of acetonitrile to directly extract the newly synthesized S1P. The amount of S1P in the samples is then quantified as follows. C₁₇ base D-erythro-sphingosine and C₁₇ S1P are used as internal standards for sphingosine and S1P, respectively. These seventeen-carbon fatty acid-linked sphingolipids are not naturally produced, making these analogs excellent standards. The lipids are then fractionation by High-Performance Liquid Chromatography using a C8-reverse phase column eluted with 1 mM methanolic ammonium formate/2 mM aqueous ammonium formate. A Finnigan LCQ Classic LC-MS/MS is used in the multiple reaction monitoring positive ionization mode to acquire ions at m/z of 300 (precursor ion)→282 (product ion) for sphingosine and 380→264 for S1P. Calibration curves are generated by plotting the peak area ratios of the synthetic standards for each sphingolipid, and used to determine the normalized amounts of sphingosine and S1P in the samples.

A “pharmaceutical composition” refers to a mixture of one or more of the compounds described herein, or pharmaceutically acceptable salts thereof, with other chemical components, such as physiologically acceptable carriers and excipients. The purpose of a pharmaceutical composition is to facilitate administration of a compound to an organism.

The term “pharmaceutically acceptable salt” refers to those salts that retain the biological effectiveness of the parent compound. Such salts include: (1) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid, or malonic acid and the like, preferably hydrochloric acid or (L)-malic acid; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, tromethamine, N-methylglucamine, and the like.

As used herein, the term a “physiologically acceptable carrier” refers to a carrier or diluent that does not cause significant irritation to an organism and does not abrogate the biological activity and properties of the administered compound. Typically, this includes those properties and/or substances that are acceptable to the patient from a pharmacological/toxicological point of view and to the manufacturing pharmaceutical chemist from a physical/chemical point of view regarding composition, formulation, stability, patient acceptance and bioavailability.

An “excipient” refers to an inert substance added to a pharmaceutical composition to further facilitate administration of a compound. Example, without limitations, of excipients include calcium carbonate, calcium phosphate, various sugars and types of starch, cellulose derivatives (including microcrystalline cellulose), gelatin, vegetable oils, polyethylene glycols, diluents, granulating agents, lubricants, binders, disintegrating agents, and the like.

The term “therapeutically effective amount” as used herein refers to that amount of the compound being administered that is effective to reduce or lessen at least one symptom of the disease being treated or to reduce or delay onset of one or more clinical markers or symptoms of the disease. In reference to the treatment of cancer, a therapeutically effective amount refers to that amount that has the effect of: (1) reducing the size of the tumor, (2) inhibiting, i.e. slowing to some extent, preferably stopping, tumor metastasis, (3) inhibiting, i.e. slowing to some extent, preferably stopping, tumor growth, and/or (4) relieving to some extent, preferably eliminating, one or more symptoms associated with the cancer.

The compounds of this invention may also act as a prodrug. The term “prodrug” refers to an agent which is converted into the parent drug in vivo. Prodrugs are often useful because, in some situations, they may be easier to administer than the parent drug. They may, for example, be bioavailable by oral administration whereas the parent drug is not. The prodrug may also have improved solubility in pharmaceutical compositions over the parent drug. An example, without limitation, of a prodrug would be a compound of the present invention which is administered as an ester (the “prodrug”), carbamate or urea.

The compounds of this invention may also be metabolized by enzymes in the body of the organism, such as a human being, to generate a metabolite that can modulate the activity of SK.

While the invention has been particularly shown and described with reference to preferred embodiments thereof, it will be understood by those skilled in the art that various alterations in form and detail may be made therein without departing from the spirit and scope of the invention. In particular, the specific method of use of the SK inhibitory compounds and compositions can vary significantly without departing from the discovered methods. Moreover, other sphingosine kinase inhibitors can be used. Preferred sphingosine kinase inhibitors are compounds that cause greater than 25% inhibition of sphingosine kinase activity in the target tissue at doses that can be obtained in an animal. In other embodiments of the invention, the sphingosine kinase inhibitor has an IC₅₀ of less than 100 μM, as measured by the LC/MS/MS assay described above. In other embodiments of the invention, the sphingosine kinase inhibitor has at least 10%, at least 20%, or yen at least 50% inhibition of recombinant SK, as measured by the technique described in Example 15, below.

Additionally, methods for the treatment of additional diseases that involve undesired ischemia-reperfusion injury occurring within particular cells of the patient are considered to be within the scope of the following claims.

The Examples, which follow, are illustrative of specific embodiments of the invention, and various uses thereof. They are set forth for explanatory purposes only, and are not to be taken as limiting the invention.

### EXAMPLES

### Example 1

ABC294640 reduces kidney damage following ischemia-reperfusion. Male C57/Bl6 mice (approximately 24 g) were first dosed with ABC294640 (50 mg/kg in 0.1 mL by oral gavage) or vehicle (0.1 mL in 0.375% Tween 80 in Phosphate-Buffered Saline), immediately followed by intraperitoneal injection of ketamine/xylazine for anesthesia. The procedure was performed on a heated surface using homeothermic pads to ensure the maintenance of animal body temperature. A midline incision was made and the two renal pedicles were located and clamped for 22 minutes or 25 minutes as indicated. Total blockage of the renal pedicle and thus artery was confirmed after several minutes as the kidney were seen to be dark red to purple in color, assuring correct clamp placement. After the scheduled time elapsed, the clamps were removed and the kidneys were observed to ensure reperfusion as indicated by returning to their original color. One milliliter of pre-warmed (37° C.) sterile saline was instilled into the peritoneum at the time of closing using sutures for musculature and wound clips for the skin incision. Animals were maintained on homeothermic pads until awakening from anesthesia and post-operatively assessed for health.

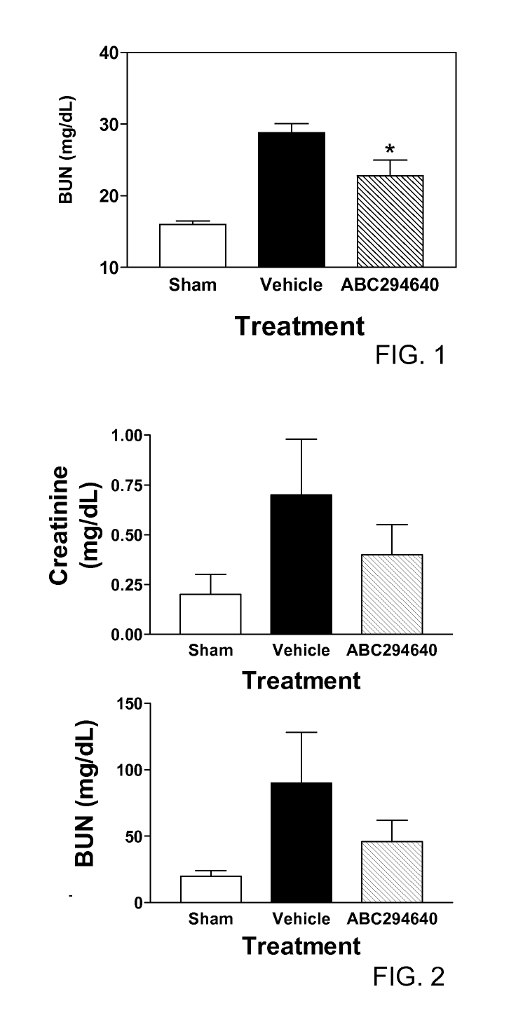

Levels of blood urea nitrogen (BUN) were determined as an indicator of renal function. As shown in FIG. 1, ischemia-reperfusion resulted in large increases in BUN levels in vehicle-treated mice (filled bar) compared with literature values of sham (non-ischemic) animals of this model (open bar). Mice treated with ABC294640 (cross-hatched bars) had significantly lower BUN levels at 72 hours after reperfusion than did control mice that received the drug vehicle alone (* indicates p<0.01). Therefore, the SK inhibitor substantially improves kidney function that persists for at least 3 days after drug administration.

### Example 2

ABC294640 reduces kidney damage following ischemia-reperfusion. Kidney ischemia-reperfusion was repeated as in Example 1, except that the duration of bilateral pedicle clamping was 25 minutes and animals were sacrificed 24 hours after surgery. As shown in FIG. 2, ischemia-reperfusion resulted in large increases in creatinine (left panel) and BUN (right panel) levels in vehicle-treated mice (filled bar) compared with literature values of sham (non-ischemic) animals of this model (open bar). Mice treated with ABC294640 (cross-hatched bars) had substantially lower creatinine and BUN levels at 24 hours after reperfusion than did control mice that received the drug vehicle alone.

### Example 3

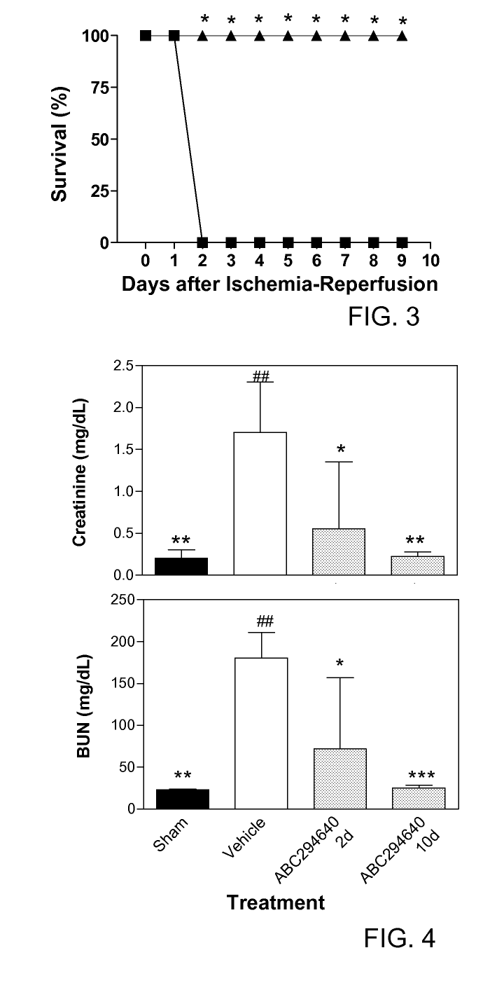

ABC294640 promotes survival in a severe model of kidney damage. To evaluate the protective effects of ABC294640 in a more severe IR model, mice were treated with ABC294640 (50 mg/kg in 0.1 mL by oral gavage) or vehicle (0.1 ml in 0.375% Tween-80 in PBS), and then the right kidney was ligated and removed and the left kidney pedicle was clamped for 45 min before reperfusion. Animals were assessed daily for postoperative health, including scoring for weight loss, activity and appearance. Following severe IR, vehicle-treated mice consistently died or were sacrificed because of severe ill health on post-surgery Day 2 (FIG. 3). In marked contrast, mice that received 50 mg/kg ABC294640 preoperatively all survived the IR insult. The ABC294640-treated animals appeared robust and were gaining weight and thus assumed to have fully recovered when sacrificed on post-surgery Day 9.

### Example 4

ABC294640 protects against renal failure in a severe model of kidney damage. Blood was drawn from the severe IR mice at sacrifice, and levels of blood urea nitrogen (BUN) and creatinine were determined as indicators of renal function (FIG. 4). As expected, serum creatinine and BUN levels were highly elevated in the vehicle-treated IR animals (clear bars) compared with sham animals (hatched bars), indicative of post-surgical renal failure. In contrast, animals treated with ABC294640 (blue bars) had significantly reduced serum creatinine and BUN levels at 48 hr post-surgery, with values returning to normal by post-surgery Day 10.

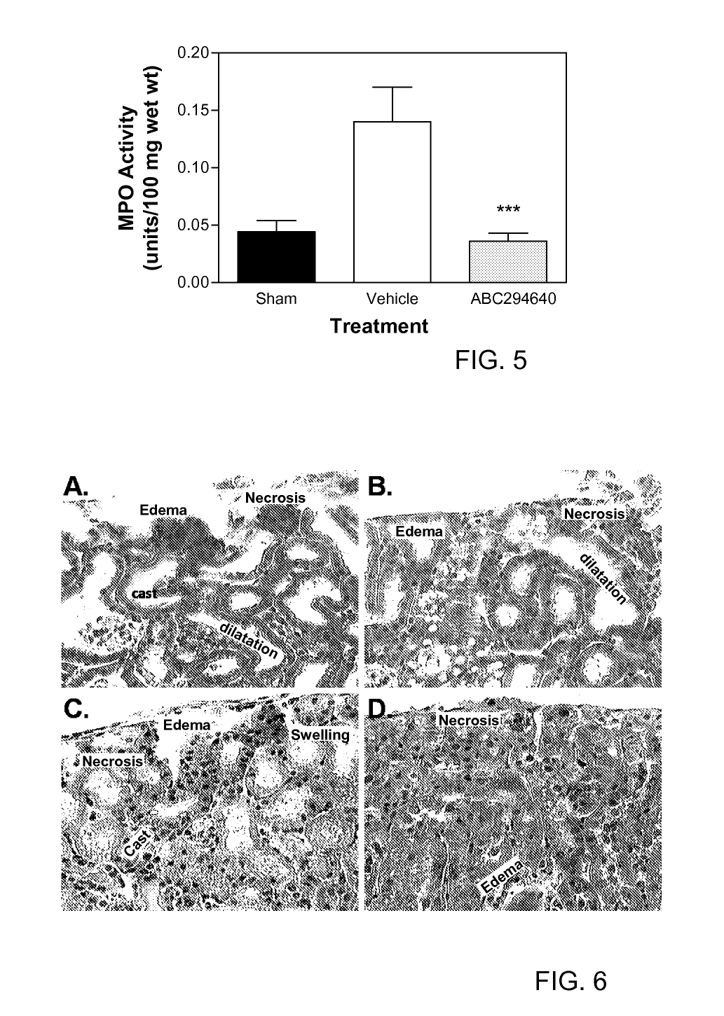

Myeloperoxidase (MPO) activity, which is reflective of neutrophil influx into the tissue, is often used as measure of local inflammation, and was assayed in the kidneys of the mice from FIG. 4. As shown in FIG. 5, MPO activity was elevated in vehicle-treated IR animals compared to sham controls. The increase in MPO activity was completely blocked in mice receiving ABC294640. Therefore, the level of kidney MPO and expected inflammatory damage in vehicle-treated animals correlates with the observed reduction in renal function.

Kidneys from the animals described with respect to FIG. 4 were sectioned and stained with H&E for histological evaluation. FIG. 6 shows representative kidney sections from animals treated with vehicle and ABC294640 at 4 and 48 hours post ischemia and reperfusion (panel A: Vehicle, 4 hours, panel B: ABC294640, 4 hours, Panel C: Vehicle, 48 hours and Panel D: ABC294640, 48 hours). Kidneys from animals treated with ABC294640 had much less damage than kidneys from vehicle treated animals at both time points. Kidney sections were quantitatively scored on a scale of 1 to 3 for five parameters (tubule cell swelling, tubular dilatation, edema, epithelial necrosis and tubular casts) that were added to generate a total score (FIG. 6).

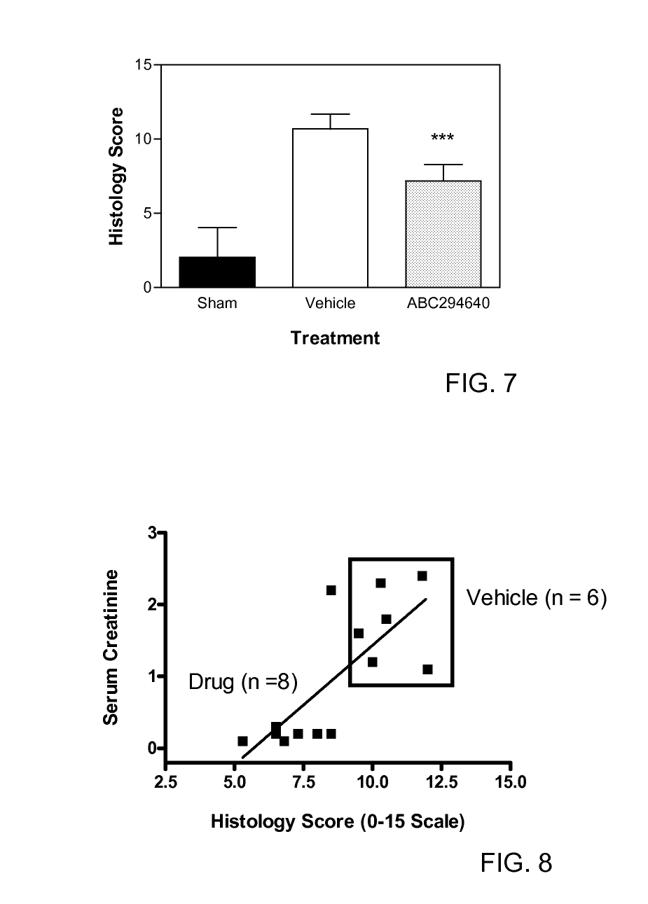

Histology scores from FIG. 7 were then paired with serum creatinine levels to relate how the changes seen in histology related with kidney function. A strong correlation was observed as demonstrated in FIG. 8.

### Example 5

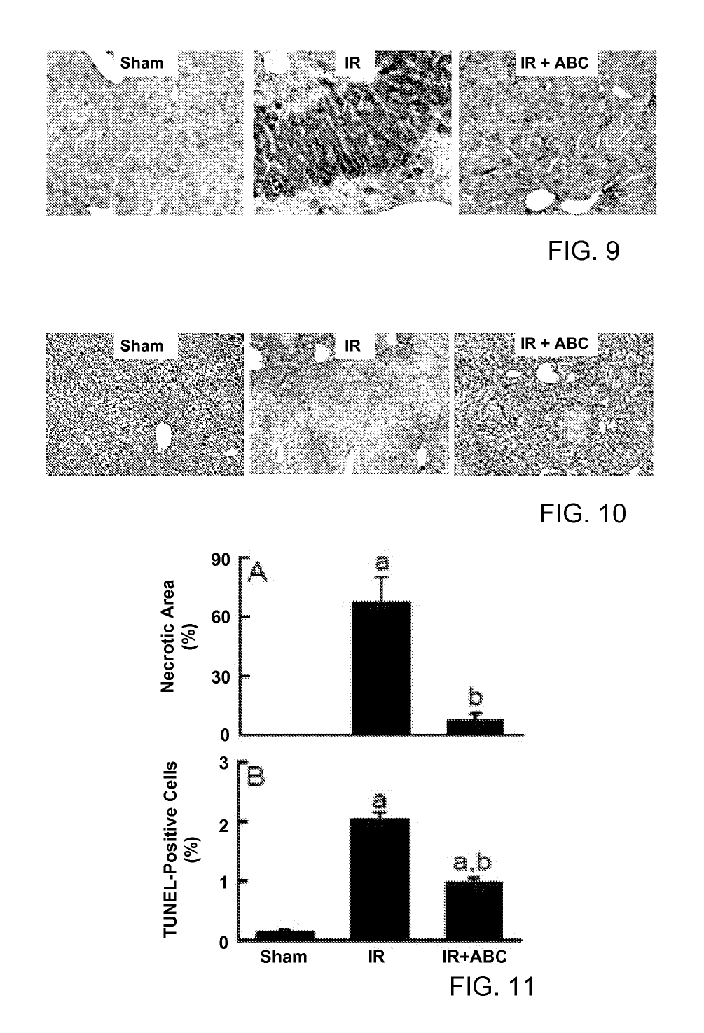

Warm IR upregulates SK in the liver. IR injury to the liver occurs in LT. Since SK is associated with inflammatory processes, we investigated whether IR affects SK expression in the liver. Male C57BL/6 mice (8-9 weeks) were gavaged with ABC294640 (ABC, 50 mg/kg) or an equal volume of vehicle (0.375% Tween-80 in phosphate buffer) 1 h prior to surgery. Under ether anesthesia, hepatic ischemia was induced by clamping the hepatic artery and portal vein to the upper three lobes of the liver (i.e., about 70% of total liver). One hour later, the ischemic liver was reperfused by opening the vascular clamp. Livers were collected 6 h later under pentobarbital anesthesia (80 mg/kg, i.p.) and SK was detected immunohistochemically. Basal levels of SK were observed in both parenchymal and non-parenchymal cells in sham-operated livers, especially in sinusoidal lining cells (FIG. 9 left panel). SK expression increased markedly after hepatic IR (FIG. 9 middle). Upregulation of SK occurred most overtly in hepatocytes in the midzonal regions of the liver lobule. ABC294640 attenuated upregulation of SK after hepatic warm IR (FIG. 9 right). These results are consistent with recent demonstrations that SK expression is induced in hypoxic cells in culture (Ader, Brizuela et al. 2008; Anelli, Gault et al. 2008).

### Example 6

ABC294640 prevents hepatic warm IR-induced cell death. SK increased after hepatic IR, therefore, we investigated whether ABC294640 protects against hepatic IR injury. No pathological changes were observed in liver tissue after sham operation (FIG. 10, left). Necrotic areas became panlobular after 1 h-ischemia plus 6 h of reperfusion (FIG. 10, middle). Necrotic area, quantified by computerized image analysis of 10 random fields per slide, accounted for 68% of the liver tissue (FIG. 11A). After ABC294640 treatment, necrosis was decreased to ˜7%, which represents an almost 90% attenuation as compared to the vehicle-treated livers (FIG. 11A). Hepatic apoptosis was evaluated by terminal deoxylnucleotidyl transferase-mediated deoxyuridine triphosphate biotin nick end labeling (TUNEL) (FIG. 11B). TUNEL-positive cells were rare in livers from sham-operated mice (0.13 cells/high power field (hpf)). TUNEL staining increased slightly to 2 cells/hpf after 1 h-ischemia plus 6 h-reperfusion (FIG. 11B). This small increase of TUNEL was partially blocked by ABC294640 (FIG. 11B). Together, the data demonstrate that warm IR causes massive cell death in the liver, and the predominant form of cell death is necrosis. Inhibition of SK effectively prevents hepatic cell death, suggesting that production of S1P plays an important role in hepatotoxicity after liver IR.

### Example 7

ABC294640 improves liver function and survival after hepatic warm IR. Liver warm ischemia was induced as described above. Blood was collected at 6 h after reperfusion, and serum alanine aminotransferase (ALT) activity and bilirubin were measured. Before ischemia, serum ALT levels were 22 U/L. At 6 h after reperfusion, ALT levels increased to ˜19,000 U/L in livers exposed to 1 h-ischemia (FIG. 12A). Pretreatment of the animals with ABC294640 decreased ALT levels after IR to ˜1,600 U/L, which reflects a >90% decrease compared to livers without ABC294640 treatment (FIG. 12A). These results indicate that ABC294640 markedly decreases hepatic IR injury. Bilirubin accumulates in the blood when liver function is poor. Serum total bilirubin was 0.26 mg/dL in mice subjected to the sham operation. Bilirubin increased 3-fold 6 h after reperfusion (FIG. 12B). ABC294640 treatment almost completely reversed the accumulation of bilirubin after warm IR (FIG. 12B).

To evaluate the effects of ABC294640 on survival of mice after IR, the non-ischemic liver lobes were removed after the vascular clamp was opened and mice were observed 7 days for survival. This procedure mimics total LT. All mice survived after sham operation (data not shown). Survival decreased to 28% after 1 h-ischemia plus reperfusion (FIG. 12C). Death occurred mainly in the first 36 h after reperfusion (FIG. 12C). Importantly, in ABC294640-pretreated mice, survival was 100% after IR (FIG. 12C), indicating that ABC294640 completely prevented acute liver failure after IR.

### Example 8

ABC294640 prevents mitochondrial depolarization after hepatic IR. MPT onset is an important mechanism leading to cell death due to mitochondrial depolarization. Our previous studies show that the MPT occurs after warm IR and LT. To determine if ABC294640 prevents mitochondrial depolarization after hepatic IR in vivo, we performed intravital multiphoton fluorescent microscopy to image living liver mitochondria. There are two main advantages of multiphoton microscopy: 1) Red/infrared light penetrates deeper than visible light into solid tissues allowing visualization of tissue planes as deep as 1 mm into thick specimens. 2) Photobleaching and photodamage are limited to the in-focus optical slice and do not occur in the remaining tissue as is the case for conventional confocal and widefield microscopy. Therefore, the viability of thick living specimens is maintained much longer with multiphoton microscopy (Lemasters 2000). These advantages make multiphoton microscopy a powerful tool for studying mitochondrial function in live animals.

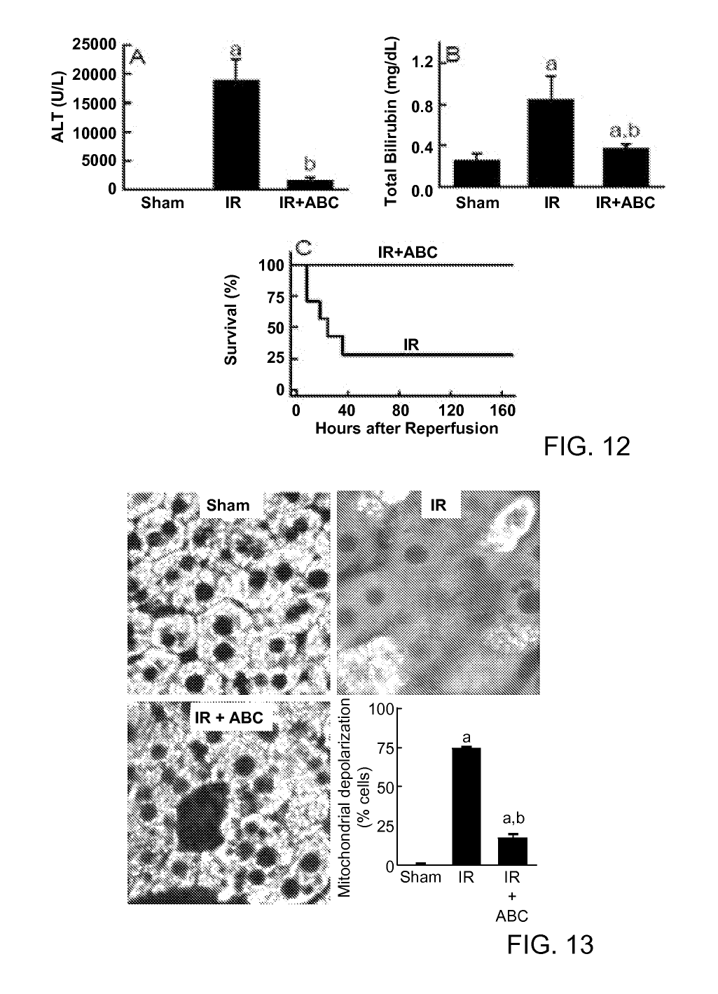

Following 1 h-ischemia and 2 h-ischemia, rhodamine-123 (Rh123), a cationic fluorophore that is taken up by polarized mitochondria, and propidium iodine (PI) that labels the nuclei of non-viable cells were infused and intravital multiphoton imaging of livers was performed. In sham-operated mice, green Rh123 fluorescence was punctate in virtually all hepatocytes, indicating mitochondrial polarization (FIG. 13. upper left). Red PI staining in nuclei was negligible. By contrast, mitochondria in many hepatocytes did not take up Rh123 at this time point (FIG. 13, upper right), indicating occurrence of widespread mitochondrial depolarization. At 2 h after IR, mitochondria of 74% of hepatocytes did not take up Rh123 (FIG. 13, lower right). Despite the absence of mitochondrial polarization, the majority of hepatocytes maintained membrane integrity as indicated by lack of nuclear red PI staining. Only ˜2% of parenchymal and nonparenchymal cells took up PI (data not shown) at this time point. Importantly, mitochondrial depolarization was rare in the livers of ABC294640-treated mice (FIG. 13. lower left). Mitochondrial depolarization only occurred in 17% of hepatocyte in ABC294640-treated mice exposed to IR (FIG. 13. lower right). These results indicated that at 2 h after hepatic IR, mitochondrial depolarization had occurred in most hepatocytes and that this event preceded hepatocyte death. ABC294640 largely prevented mitochondrial depolarization after IR.

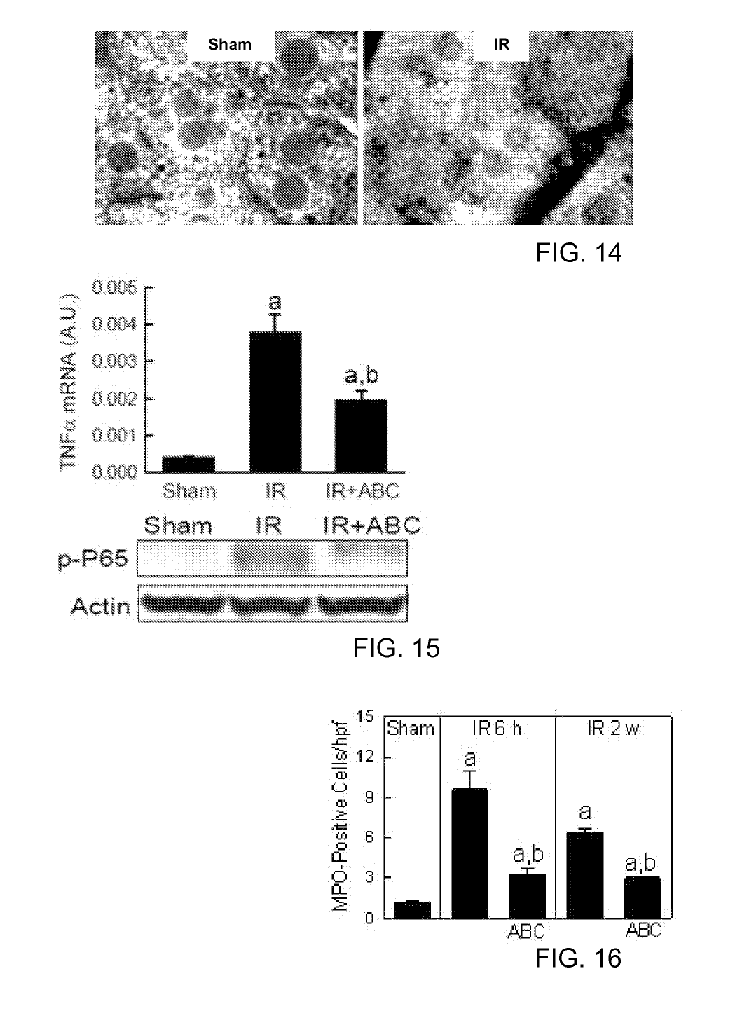

To investigate whether mitochondrial depolarization is caused by MPT onset, intravital confocal/multiphoton imaging of calcein was performed. Calcein, a fluorophore that loads into the cytosol, outlined mitochondria as dark voids in the hepatocytes from sham-operated mice (FIG. 14. left). These voids disappeared at 2 h after reperfusion (FIG. 14. right). Calcein gains entrance to the mitochondrial matrix space only when PT pores open. Therefore, disappearance of voids indicates MPT onset. This finding shows directly that the MPT occurs in vivo and is a sequel of IR insult to the liver.

### Example 9

ABC294640 prevents hepatic warm IR-induced tumor necrosis factor-α (TNFα) formation and NF-κB activation. Toxic cytokine formation and inflammatory processes play important roles in IR injury, and S1P is well known to promote inflammation. Accordingly, we investigated whether ABC294640 affects the expression of the pro-inflammatory cytokine TNFα after IR. Livers were harvested at 2 h after reperfusion and TNFα mRNA was detected by quantitative real time PCR. TNFα mRNA increased ˜10-fold after IR (FIG. 15, upper). ABC294640 blunted this increase in TNFα mRNA by ˜50%. NF-κB activation is involved in inflammatory cytokine formation and upregulation of adhesion molecules. After IR, phosphorylation of p65 subunit of NF-κB increased markedly, indicating NF-κB activation. This effect was also blunted by ABC294640 (FIG. 15 lower) which is consistent with our previous studies of NF-κB activation.

### Example 10

ABC294640 prevents hepatic warm ischemia-reperfusion (IR)-induced polymorphonuclear leukocyte (PMN) infiltration in mice. Hepatic warm ischemia was induced by clamping the mouse hepatic artery and portal vein to the upper three lobes of the liver as described in the original application. One hour later, the ischemic liver was reperfused by opening the vascular clamp. Livers were collected 6 h later and myeloperoxidase (MPO), a marker of PMNs, was detected immunohistochemically. MPO-positive cells were counted in 10 fields selected randomly per slide in a blinded manner to assess PMN infiltration. MPO-positive cells were ˜1/high power field (hpf) in livers from sham-operated mice (FIG. 16). Six hours after reperfusion, MPO-positive cells increased almost 10-fold, indicating pronounced inflammation. PMN infiltration remained higher than the basal level at 2 weeks after reperfusion (˜6 cells/hpf) (FIG. 16), indicating that inflammatory processes still exist long after the initial injury. ABC294640 (50 mg/kg, i.g. once) decreased PMN infiltration by ˜70% at 6 h after reperfusion (FIG. 16). Livers from mice exposed to one dose of ABC294640 had lower PMN infiltration compared to the untreated mice even at 2 weeks after reperfusion. These results indicate that ABC294640 indeed inhibits acute and chronic inflammation after hepatic warm IR.

### Example 11

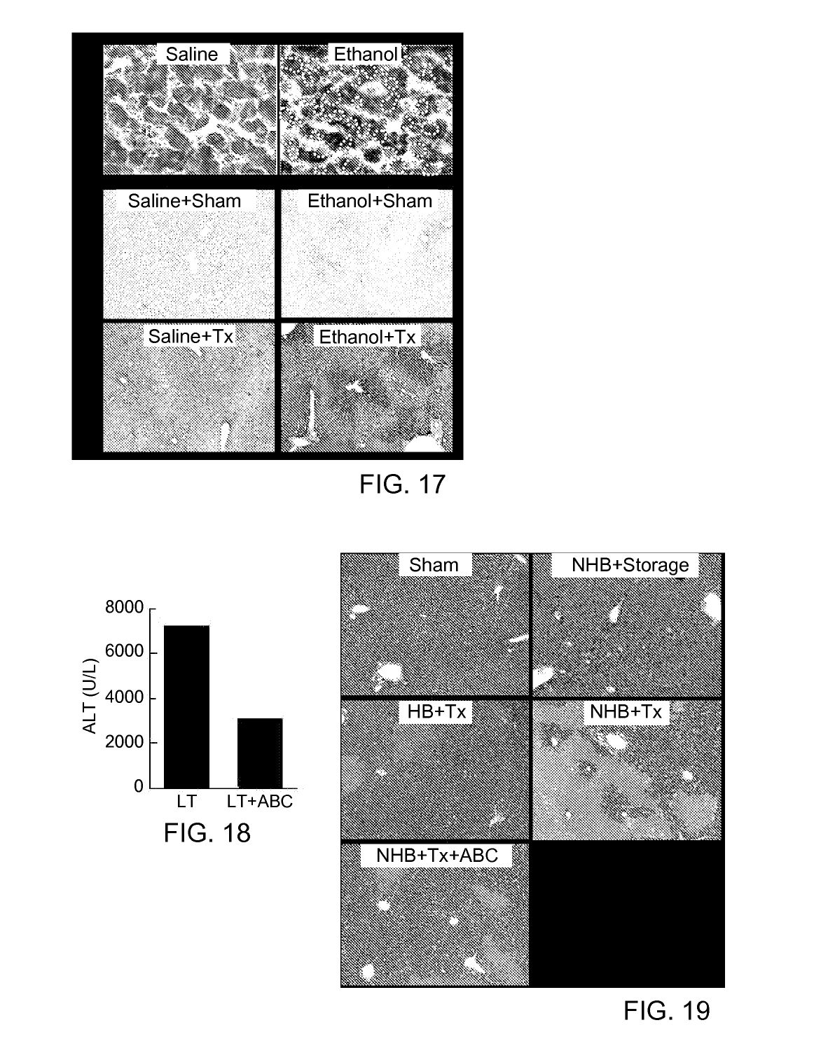

Sphingosine kinase (SK) is upregulated dramatically after fatty liver transplantation. Liver transplantation is currently limited by a severe shortage of optimal donor livers. Hepatic steatosis, which occurs in 30-50% of liver donors, increases primary nonfunction and subsequent graft failure. Organ donors are mainly accident victims where heavy alcohol consumption, a known risk factor for hepatic steatosis, is frequently involved. Previous studies have shown that both acute and chronic alcoholic hepatic steatosis increases graft failure after liver transplantation. It is unknown if SK plays a role in the failure of fatty liver grafts, so SK expression in fatty liver grafts was examined. Lewis rats were gavaged with saline or an inebriating dose of ethanol (6 g/kg), livers were harvested 20 h later and implanted after cold storage in UW solution. Liver grafts were collected 8 h after implantation and SK in liver sections was detected immunohistochemically. Ethanol treatment caused overt hepatic steatosis as detected by Oil-Red-O staining (FIG. 17). Basal levels of SK were observed in livers from saline-treated, untransplanted livers, and were not significantly altered after ethanol treatment alone (FIG. 17 upper panels). SK expression substantially increased after transplantation of lean livers from saline-treated rats (FIG. 17 lower left). Notably, SK expression increased dramatically after transplantation of fatty livers from ethanol-treated rats (FIG. 17 lower right). These results suggest that SK over-expression has an important role in fatty liver graft failure. Therefore, ABC294640 is likely to have substantial beneficial effects on fatty liver transplantation.

### Example 12

ABC294640 decreases graft injury after lean LT. To investigate whether ABC294640 protects liver grafts after LT, a pilot study was conducted. Lean livers were explanted and stored in UW solution for 8 h. ABC294640 was added to the UW solution and the lactated Ringer's post-storage solution at a concentration of 60 μM and injected into the recipients (50 mg/kg, i.p.) immediately after transplantation. Six hours after LT, serum ALT increased to 7200 U/L in vehicle-treated rats, but was markedly attenuated in ABC294640-treated rats (FIG. 18). These results suggest that ABC294640 indeed can improve the outcome of lean LT. Further studies described herein are needed to confirm these results and to optimize the dose and method of drug administration to achieve even better protection.

### Example 13

ABC294640 improves the outcome of non-heart-beating liver transplantation. The severe donor organ shortage could be reduced by the use of marginal livers for transplantation. Currently only livers from brain-dead, heart-beating donors (HBD) are used for transplantation since liver grafts from non-heart-beating donors frequently fail after transplantation. Development of a method to improve survival of grafts from NHBD is critical to expand the usable liver donor pool. Grafts from NHBD experience longer warm ischemia before liver retrieval, which likely upregulates SK to a higher extent compared to those from HBD. Therefore, we performed a pilot study to test if ABC294640 improves the outcome of non-heart-beating liver transplantation. Livers were explanted from Lewis rats after 30-min of aorta clamping to mimic non-heart-beating donation and implanted after 4-hour storage in UW solution at 0-1° C. No pathological changes were observed in livers 18 h after sham operation (FIG. 19, upper left). In cold-stored, untransplanted livers from NHBD, although some cell swelling was observed, no necrosis occurred (FIG. 19, upper right). After transplantation of livers from HBD, small focal necrosis occurred (FIG. 19, middle left). In contrast, after transplantation of liver grafts from NHBD, large area of necrosis were observed (FIG. 19, middle right). Inhibition of SK with ABC294640 substantially decreased necrotic areas within the transplanted livers (FIG. 19, lower left). These results suggest that ABC294640 can be used not only to improve the outcome of transplantation of healthy liver grafts, but also to improve the outcome of marginal liver transplantation.

### Example 14

Additional examples of sphingosine kinase inhibitors suitable for use in the methods of the present invention are provided in the tables below.

TABLE 1
Representative adamantane-based compounds

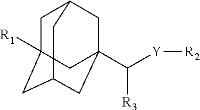

| Cmpd | Chemical name | Y | R₃ | R₁ | R₂ |
|---|---|---|---|---|---|
| A1 | 3-(4-Chloro-phenyl)-adamantane-1- carboxylic acid isopropylamide | NH | ═O | 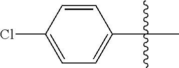 | 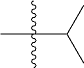 |
| A2 | 3-(4-Chloro-phenyl)-adamantane-1- carboxylic acid cyclopropylamide | NH | ═O | 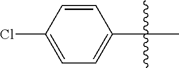 | 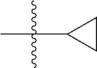 |
| A3 | 3-(4-Chloro-phenyl)-adamantane-1- carboxylic acid (2-ethylsulfanyl- ethyl)-amide | NH | ═O | 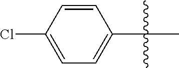 | 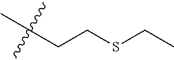 |
| A4 | 3-(4-Chloro-phenyl)-adamantane-1- carboxylic acid phenylamide | NH | ═O | 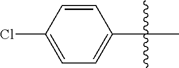 | 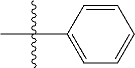 |
| A5 | Adamantane-1-carboxylic acid (4- hydroxy-phenyl)-amide | NH | ═O | H | 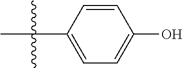 |
| A6 | 3-(4-Chloro-phenyl)-adamantane-1- carboxylic acid (4-hydroxy-phenyl)- amide | NH | ═O | 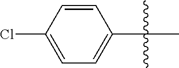 | 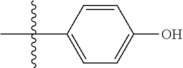 |
| A7 | Acetic acid 4-{[3-(4-chloro-phenyl)- adamantane-1-carbonyl]-amino}- phenyl ester | NH | ═O | 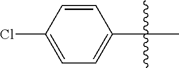 | 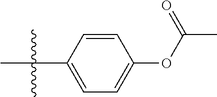 |
| A8 | 3-(4-Chloro-phenyl)-adamantane-1- carboxylic acid (2,4-dihydroxy- phenyl)-amide | NH | ═O | 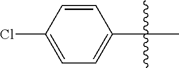 | 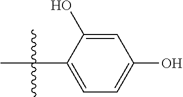 |
| A9 | 3-(4-Chloro-phenyl)-adamantane-1- carboxylic acid (3-hydroxymethyl- phenyl)-amide | NH | ═O | 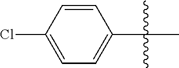 | 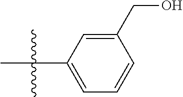 |
| A10 | Adamantane-1-carboxylic acid (4- cyanomethyl-phenyl)-amide | NH | ═O | H | 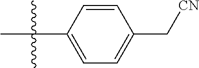 |
| A11 | 3-(4-Chloro-phenyl)-adamantane-1- carboxylic acid (4-cyanomethyl- phenyl)-amide | NH | ═O | 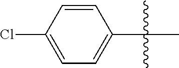 | 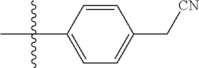 |
| A12 | 3-(4-Chloro-phenyl)-adamantane-1- carboxylic acid benzylamide | NH | ═O | 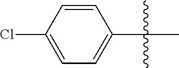 | 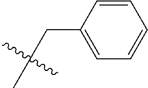 |
| A13 | 3-(4-Chloro-phenyl)-adamantane-1- carboxylic acid 4-tert-butyl- benzylamide | NH | ═O | 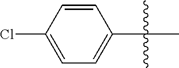 | 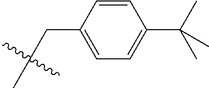 |
| A14 | 3-(4-Chloro-phenyl)-adamantane-1- carboxylic acid 4-methylsulfanyl- benzylamide | NH | ═O | 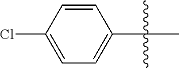 | 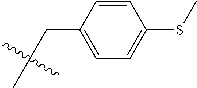 |
| A15 | 3-(4-Chloro-phenyl)-adamantane-1- carboxylic acid 3-trifluoromethyl- benzylamide | NH | ═O | 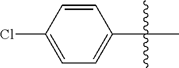 | 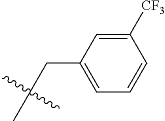 |
| A16 | 3-(4-Chloro-phenyl)-adamantane-1- carboxylic acid 4-trifluoromethyl- benzylamide | NH | ═O | 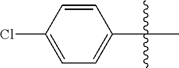 | 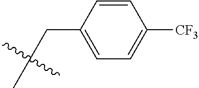 |
| A17 | 3-(4-Chloro-phenyl)-adamantane-1- carboxylic acid 3,5-bis- trifluoromethyl-benzylamide | NH | ═O | 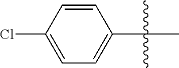 | 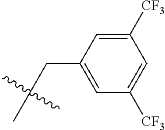 |
| A18 | 3-(4-Chloro-phenyl)-adamantane-1- carboxylic acid 3-fluoro-5- trifluoromethyl-benzylamide | NH | ═O | 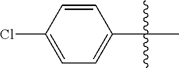 | 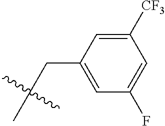 |
| A19 | 3-(4-Chloro-phenyl)-adamantane-1- carboxylic acid 2-fluoro-4- trifluoromethyl-benzylamide | NH | ═O | 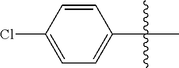 | 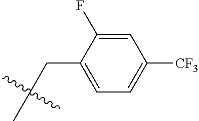 |
| A20 | 3-(4-Chloro-phenyl)-adamantane-1- carboxylic acid 3,5-difluoro- benzylamide | NH | ═O | 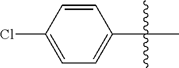 | 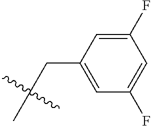 |
| A21 | 3-(4-Chloro-phenyl)-adamantane-1- carboxylic acid 3,4-difluoro- benzylamide | NH | ═O | 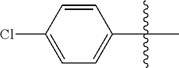 | 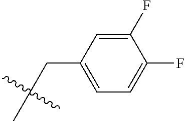 |
| A22 | 3-(4-Chloro-phenyl)-adamantane-1- carboxylic acid 3,4,5-trifluoro- benzylamide | NH | ═O | 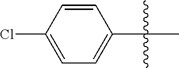 | 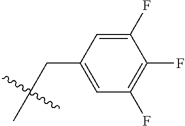 |
| A23 | 3-(4-Chloro-phenyl)-adamantane-1- carboxylic acid 3-chloro-4-fluoro- benzylamide | NH | ═O | 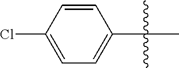 | 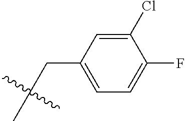 |
| A24 | 3-(4-Chloro-phenyl)-adamantane-1- carboxylic acid 4-fluoro-3- trifluoromethyl-benzylamide | NH | ═O | 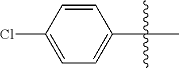 | 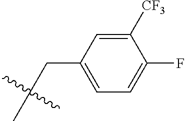 |
| A25 | 3-(4-Chloro-phenyl)-adamantane-1- carboxylic acid 2-chloro-4-fluoro- benzylamide | NH | ═O | 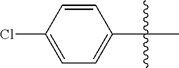 | 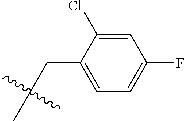 |
| A26 | 3-(4-Chloro-phenyl)-adamantane-1- carboxylic acid 4-chloro-3- trifluoromethyl-benzylamide | NH | ═O | 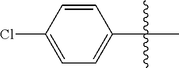 | 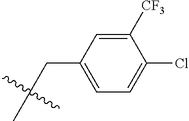 |
| A27 | 3-(4-Chloro-phenyl)-adamantane-1- carboxylic acid 3-aminomethyl- 2,4,5,6-tetrachloro-benzylamide | NH | ═O | 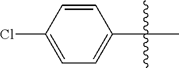 | 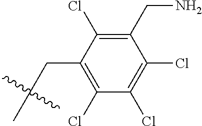 |
| A28 | 3-(4-Chloro-phenyl)-adamantane-1- carboxylic acid [1-(4-chloro- phenyl)-ethyl]-amide | NH | ═O | 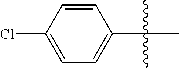 | 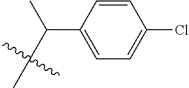 |
| A29 | 3-(4-Chloro-phenyl)-adamantane-1- carboxylic acid [1-(4-bromo- phenyl)-ethyl]-amide | NH | ═O | 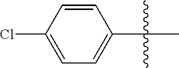 | 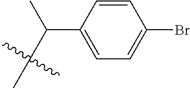 |
| A30 | 3-(4-Chloro-phenyl)-adamantane-1- carboxylic acid 4-methanesulfonyl- benzylamide | NH | ═O | 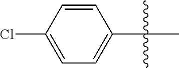 | 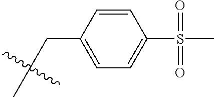 |
| A31 | 3-(4-Chloro-phenyl)-adamantane-1- carboxylic acid 4-dimethylamino- benzylamide | NH | ═O | 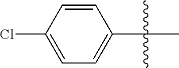 | 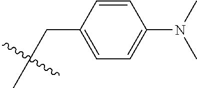 |
| A32 | 3-(4-Chloro-phenyl)-adamantane-1- carboxylic acid 4-trifluoromethoxy- benzylamide | NH | ═O | 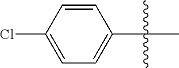 | 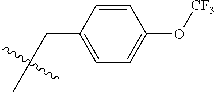 |
| A33 | 3-(4-Chloro-phenyl)-adamantane-1- carboxylic acid 3-trifluoromethoxy- benzylamide | NH | ═O | 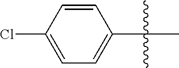 | 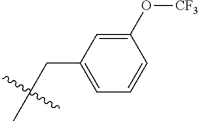 |
| A34 | 3-(4-Chloro-phenyl)-adamantane-1- carboxylic acid 4-phenoxy- benzylamide | NH | ═O | 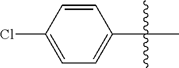 | 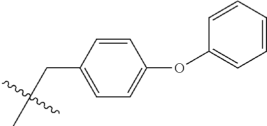 |
| A35 | Adamantane-1-carboxylic acid 3,4- dihydroxy-benzylamide | NH | ═O | H | 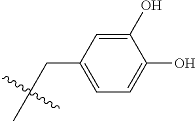 |
| A36 | 3-(4-Chloro-phenyl)-adamantane-1- carboxylic acid 3,4-dihydroxy- benzylamide | NH | ═O | 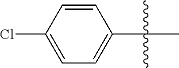 | 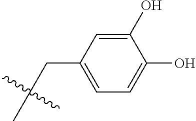 |
| A37 | 3-(4-Chloro-phenyl)-adamantane-1- carboxylic acid phenethyl-amide | NH | ═O | 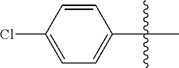 | 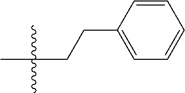 |
| A38 | 3-(4-Chloro-phenyl)-adamantane-1- carboxylic acid [2-(4-fluoro-phenyl)- ethyl]-amide | NH | ═O | 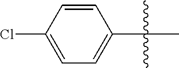 | 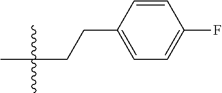 |
| A39 | 3-(4-Chloro-phenyl)-adamantane-1- carboxylic acid [2-(4-bromo- phenyl)-ethyl]-amide | NH | ═O | 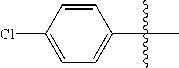 | 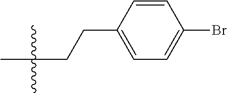 |
| A40 | 3-(4-Chloro-phenyl)-adamantane-1- carboxylic acid [2-(4-hydroxy- phenyl)-ethyl]-amide | NH | ═O | 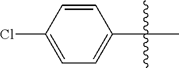 | 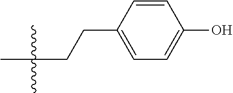 |
| A41 | 3-(4-Chloro-phenyl)-adamantane-1- carboxylic acid 4-phenoxy- benzylamide | NH | ═O | 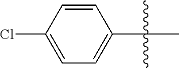 | 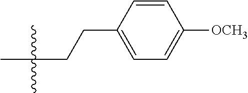 |
| A42 | 3-(4-Chloro-phenyl)-adamantane-1- carboxylic acid [2-(3-bromo-4- methoxy-phenyl)-ethyl]-amide | NH | ═O | 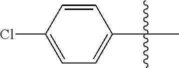 | 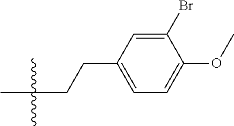 |
| A43 | Adamantane-1-carboxylic acid [2- (3,4-dihydroxy-phenyl)-ethyl]-amide | NH | ═O | H | 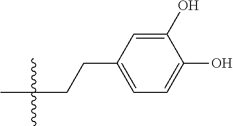 |
| A44 | 3-(4-Chloro-phenyl)-adamantane-1- carboxylic acid [2-(3,4-dihydroxy- phenyl)-ethyl]-amide | NH | ═O | 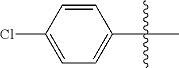 | 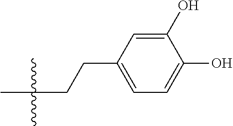 |
| A45 | 3-(4-Chloro-phenyl)-adamantane-1- carboxylic acid (2-benzo[1,3]dioxol- 5-yl-ethyl)-amide | NH | ═O | 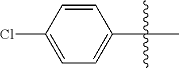 | 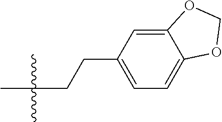 |
| A46 | 3-(4-Chloro-phenyl)-adamantane-1- carboxylic acid [2-(3-phenoxy- phenyl)-ethyl]-amide | NH | ═O | 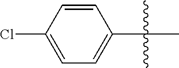 | 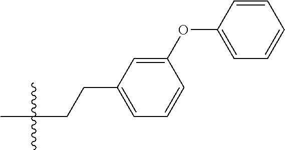 |
| A47 | 3-(4-Chloro-phenyl)-adamantane-1- carboxylic acid [2-(4-phenoxy- phenyl)-ethyl]-amide | NH | ═O | 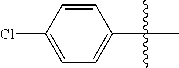 | 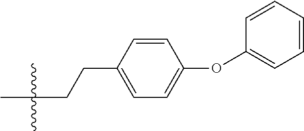 |
| A48 | 3-(4-Chloro-phenyl)-adamantane-1- carboxylic acid (3-phenyl-propyl)- amide | NH | ═O | 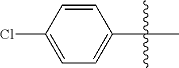 | 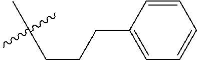 |
| A49 | 3-(4-Chloro-phenyl)-adamantane-1- carboxylic acid (biphenyl-4- ylmethyl)-amide | NH | ═O | 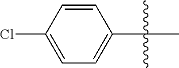 | 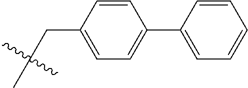 |
| A50 | Adamantane-1-carboxylic acid (1- methyl-piperidin-4-yl)-amide | NH | ═O | H | 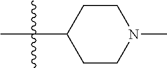 |
| A51 | 3-(4-Chloro-phenyl)-adamantane-1- carboxylic acid (1-methyl-piperidin- 4-yl)-amide | NH | ═O | 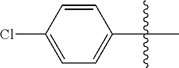 | 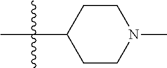 |
| A52 | 3-(4-Chloro-phenyl)-adamantane-1- carboxylic acid (4-methyl-piperazin- 1-yl)-amide | NH | ═O | 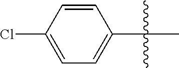 | 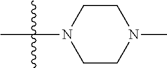 |
| A53 | 3-(4-Chloro-phenyl)-adamantane-1- carboxylic acid (3-tert-butylamino- propyl)-amide | NH | ═O | 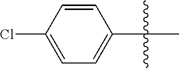 | 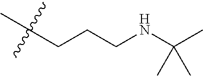 |
| A54 | 3-(4-Chloro-phenyl)-adamantane-1- carboxylic acid (3-pyrrolidin-1-yl- propyl)-amide | NH | ═O | 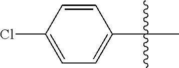 | 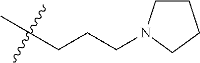 |
| A55 | 3-(4-Chloro-phenyl)-adamantane-1- carboxylic acid [3-(2-oxo-pyrrolidin- 1-yl)-propyl]-amide | NH | ═O | 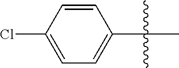 | 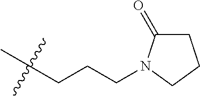 |
| A56 | Adamantane-1-carboxylic acid [2-(1- methyl-pyrrolidin-2-yl)-ethyl]-amide | NH | ═O | H | 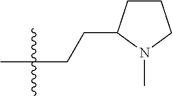 |
| A57 | 3-(4-Chloro-phenyl)-adamantane-1- carboxylic acid [2-(1-methyl- pyrrolidin-2-yl)-ethyl]-amide | NH | ═O | 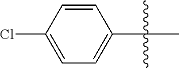 | 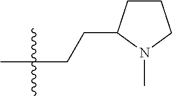 |
| A58 | 3-(4-Chloro-phenyl)-adamantane-1- carboxylic acid (2-morpholin-4-yl- ethyl)-amide | NH | ═O | 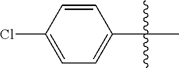 | 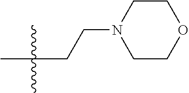 |
| A59 | 3-(4-Chloro-phenyl)-adamantane-1- carboxylic acid (2-piperazin-1-yl- ethyl)-amide | NH | ═O | 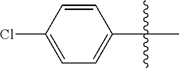 | 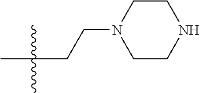 |
| A60 | Adamantane-1-carboxylic acid (pyridin-4-ylmethyl)-amide | NH | ═O | H | 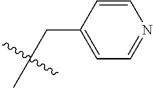 |
| A61 | 3-(4-Fluoro-phenyl)-adamantane-1- carboxylic acid (pyridin-4- ylmethyl)-amide | NH | ═O | 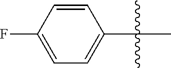 | 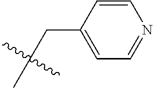 |
| A62 | 3-(4-Chloro-phenyl)-adamantane-1- carboxylic acid (pyridin-4- ylmethyl)-amide (ABC294640) | NH | ═O | 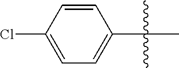 | 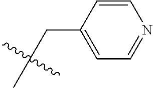 |
| A63 | Adamantane-1-carboxylic acid (pyridin-4-ylmethyl)-amide | NH | ═O | H | 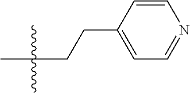 |
| A64 | 3-(4-Chloro-phenyl)-adamantane-1- carboxylic acid (2-pyridin-4-yl- ethyl)-amide | NH | ═O | 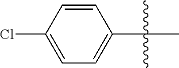 | 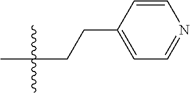 |
| A65 | 3-(4-Chloro-phenyl)-adamantane-1- carboxylic acid (3-imidazol-1-yl- propyl)-amide | NH | ═O | 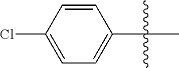 | 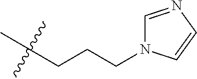 |
| A66 | 3-(4-Chloro-phenyl)-adamantane-1- carboxylic acid (2-methyl-1H-indol- 5-yl)-amide | NH | ═O | 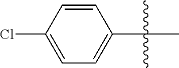 | 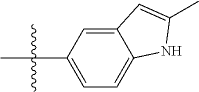 |
| A67 | 3-(4-Chloro-phenyl)-adamantane-1- carboxylic acid (1H-tetrazol-5-yl)- amide | NH | ═O | 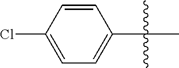 | 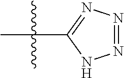 |
| A68 | 3-(4-Chloro-phenyl)-adamantane-1- carboxylic acid (9-ethyl-9H- carbazol-3-yl)-amide | NH | ═O | 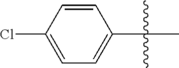 | 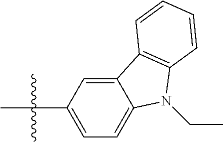 |
| A69 | Adamantane-1-carboxylic acid [4-(4- chloro-phenyl)-thiazol-2-yl]-amide | NH | ═O | H | 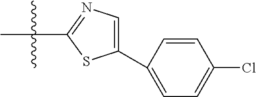 |
| A70 | 3-(4-Chloro-phenyl)-adamantane-1- carboxylic acid [4-(4-chloro- phenyl)-thiazol-2-yl]-amide | NH | ═O | 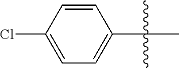 | 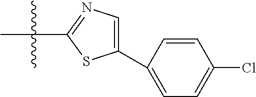 |
| A71 | 3-(4-Chloro-phenyl)-adamantane-1- carboxylic acid benzothiazol-2- ylamide | NH | ═O | 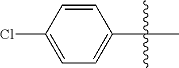 | 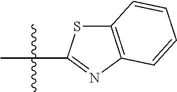 |
| A72 | 3-(4-Chloro-phenyl)-adamantane-1- carboxylic acid (5-chloro- benzooxazol-2-yl)-amide | NH | ═O | 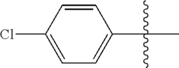 | 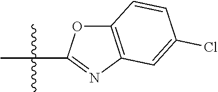 |
| A73 | 3-(4-Chloro-phenyl)-adamantane-1- carboxylic acid (9H-purin-6-yl)- amide | NH | ═O | 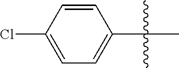 | 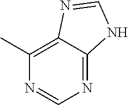 |
| A75 | [3-(4-Chloro-phenyl)-adamantan-1- ylmethyl]-isopropyl-amine | NH | H | 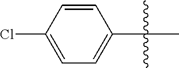 | 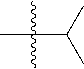 |
| A76 | 4- and -phenol | NH | H | 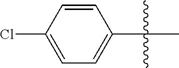 | 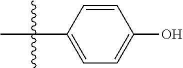 |
| A77 | [3-(4-Chloro-phenyl)-adamantan-1- ylmethyl]-(4-trifluoromethyl- benzyl)-amine | NH | H | 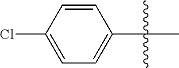 | 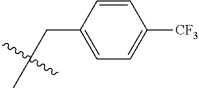 |
| A78 | [3-(4-Chloro-phenyl)-adamantan-1- ylmethyl]-(2-fluoro-4- trifluoromethyl-benzyl)-amine | NH | H | 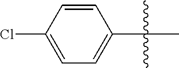 | 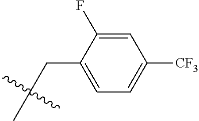 |
| A79 | [3-(4-Chloro-phenyl)-adamantan-1- ylmethyl]-(4-fluoro-3- trifluoromethyl-benzyl)-amine | NH | H | 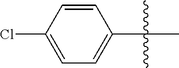 | 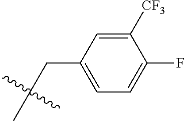 |
| A80 | [3-(4-Chloro-phenyl)-adamantan-1- ylmethyl]-(4-trifluoromethoxy- benzyl)-amine | NH | H | 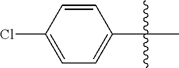 | 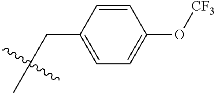 |
| A81 | [3-(4-Chloro-phenyl)-adamantan-1- ylmethyl]-[2-(3-phenoxy-phenyl)- ethyl]-amine | NH | H | 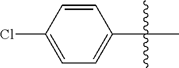 | 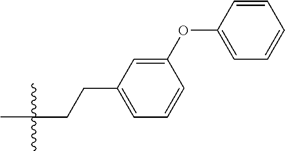 |
| A82 | [3-(4-Chloro-phenyl)-adamantan-1- ylmethyl]-(1-methyl-piperidin-4-yl)- amine | NH | H | 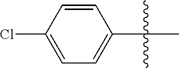 | 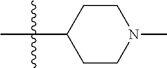 |
| A83 | [3-(4-Chloro-phenyl)-adamantan-1- ylmethyl]-(4-methyl-piperazin-1-yl)- amine | NH | H | 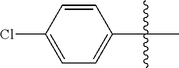 | 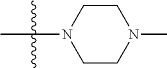 |
| A84 | N-tert-Butyl-N′-[3-(4-chloro- phenyl)-adamantan-1-ylmethyl]- propane-1,3-diamine | NH | H | 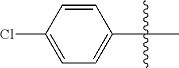 | 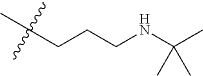 |
| A85 | [3-(4-Chloro-phenyl)-adamantan-1- ylmethyl]-(3-pyrrolidin-1-yl-propyl)- amine | NH | H | 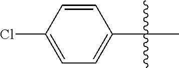 | 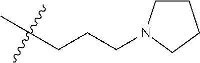 |
| A86 | [3-(4-Chloro-phenyl)-adamantan-1- ylmethyl]-[2-(1-methyl-pyrrolidin-2- yl)-ethyl]-amine | NH | H | 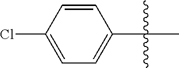 | 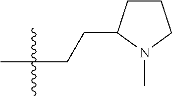 |
| A87 | [3-(4-Chloro-phenyl)-adamantan-1- ylmethyl]-(2-morpholin-4-yl-ethyl)- amine | NH | H | 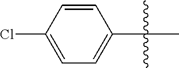 | 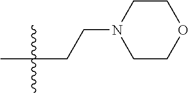 |
| A88 | [3-(4-Chloro-phenyl)-adamantan-1- ylmethyl]-pyridin-4-ylmethyl-amine | NH | H | 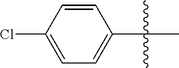 | 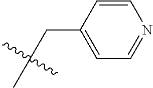 |
| A89 | [3-(4-Chloro-phenyl)-admantan-1- ylmethyl]-(9-ethyl-9H-carbazol-3- yl)-amine | NH | H | 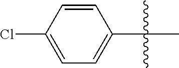 | 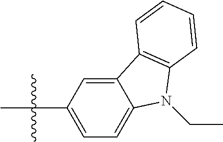 |
| A90 | [3-(4-Chloro-phenyl)-adamantan-1- ylmethyl]-[5-(4-chloro-phenyl)- thiazol-2-yl]-amine | NH | H | 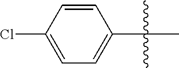 | 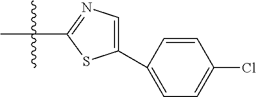 |
| A91 | 1-[3-(4-Chloro-phenyl)-adamantan- 1-yl]-ethylamine | NH | CH₃ | 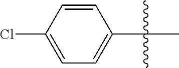 | H |
| A92 | {1-[3-(4-Chloro-phenyl)-adamantan- 1-yl]-ethyl}-isopropyl-amine | NH | CH₃ | 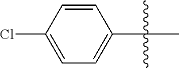 | 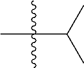 |
| A93 | Phenyl-[1-(3-phenyl-adamantan-1- yl)-ethyl]-amine | NH | CH₃ | 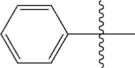 | 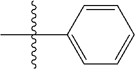 |
| A94 | {1-[3-(4-Fluoro-phenyl)-adamantan- 1-yl]-ethyl}-phenyl-amine | NH | CH₃ | 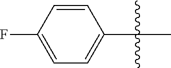 | 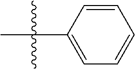 |
| A95 | {1-[3-(4-Chloro-phenyl)-adamantan- 1-yl]-ethyl}-phenyl-amine | NH | CH₃ | 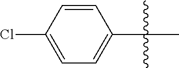 | 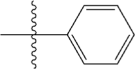 |
| A96 | (1-Adamantan-1-yl-ethyl)-benzyl- amine | NH | CH₃ | H | 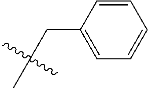 |
| A97 | Benzyl-[1-(3-phenyl-adamantan-1- yl)-ethyl]-amine | NH | CH₃ | 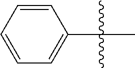 | 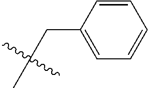 |
| A98 | Benzyl-{1-[3-(4-fluoro-phenyl)- adamantan-1-yl]-ethyl}-amine | NH | CH₃ | 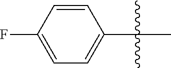 | 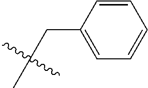 |
| A99 | Benzyl-{1-[3-(4-chloro-phenyl)- adamantan-1-yl]-ethyl}-amine | NH | CH₃ | 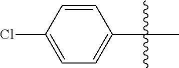 | 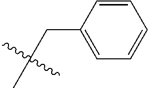 |
| A100 | (4-tert-Butyl-benzyl)-{1-[3-(4- chloro-phenyl)-adamantan-1-yl]- ethyl}-amine | NH | CH₃ | 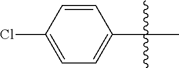 | 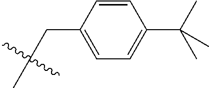 |
| A101 | [1-(4-Bromo-phenyl)-ethyl]-{1-[3- (4-chloro-phenyl)-adamantan-1-yl]- ethyl}-amine | NH | CH₃ | 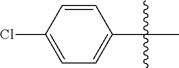 | 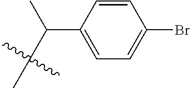 |
| A102 | (1-Adamantan-1-yl-ethyl)-[2-(4- bromo-phenyl)-ethyl]-amine | NH | CH₃ | H | 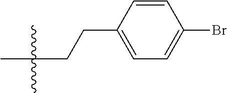 |
| A103 | [2-(4-Bromo-phenyl)-ethyl]-{1-[3- (4-chloro-phenyl)-adamantan-1-yl]- ethyl}-amine | NH | CH₃ | 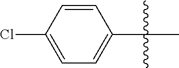 | 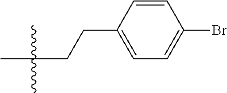 |
| A104 | (1-Adamantan-1-yl-ethyl)-(1- methyl-piperidin-4-yl)-amine | NH | CH₃ | H | 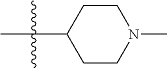 |
| A105 | (1-Methyl-piperidin-4-yl)-[1-(3- phenyl-adamantan-1-yl)-ethyl]- amine | NH | CH₃ | 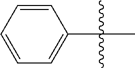 | 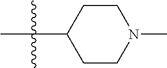 |
| A106 | {1-[3-(4-Fluoro-phenyl)-adamantan- 1-yl]-ethyl}-(1-methyl-piperidin-4- yl)-amine | NH | CH₃ | 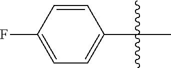 | 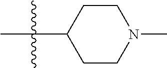 |
| A107 | {1-[3-(4-Chloro-phenyl)-adamantan- 1-yl]-ethyl}-(1-methyl-piperidin-4- yl)-amine | NH | CH₃ | 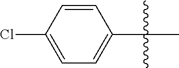 | 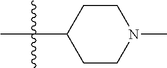 |
| A108 | {1-[3-(4-Chloro-phenyl)-adamantan- 1-yl]-ethyl}-(4-methyl-piperazin-1- yl)-amine | NH | CH₃ | 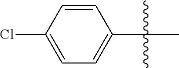 | 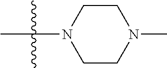 |
| A109 | {1-[3-(Phenyl)-adamantan-1-yl]- ethyl}-pyridin-4-ylmethyl-amine | NH | CH₃ | 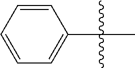 | 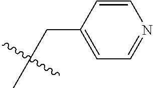 |
| A110 | {1-[3-(4-Chloro-phenyl)-adamantan- 1-yl]-ethyl}-(6-chloro-pyridin-3- ylmethyl)-amine | NH | CH₃ | 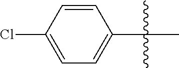 | 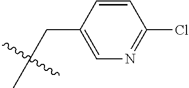 |
| A111 | {1-[3-(4-Chloro-phenyl)-adamantan- 1-yl]-ethyl}-(2-pyridin-4-yl-ethyl)- amine | NH | CH₃ | 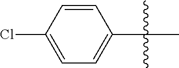 | 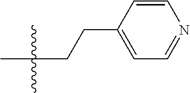 |
| A112 | {1-[3-(4-Chloro-phenyl)-adamantan- 1-yl]-ethyl}-(3H-imdazol-4- ylmethyl)-amine | NH | CH₃ | 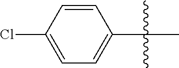 | 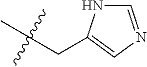 |
| A113 | {1-[3-(4-Chloro-phenyl)-adamantan- 1-yl]-ethyl}-(2-methyl-1H-indol-5- yl)-amine | NH | CH₃ | 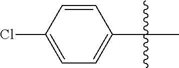 | 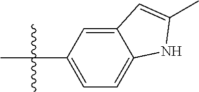 |
| A114 | {1-[3-(4-Chloro-phenyl)-adamantan- 1-yl]-ethyl}-(9-ethyl-9H-carbazol-3- yl)-amine | NH | CH₃ | 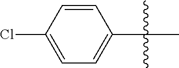 | 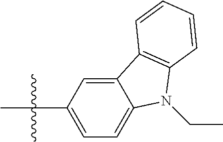 |
| A115 | {1-[3-(4-Chloro-phenyl)-adamantan- 1-yl]-ethyl}-(9-ethyl-9H-carbazol-3- ylmethyl)-amine | NH | CH₃ | 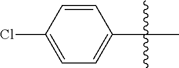 | 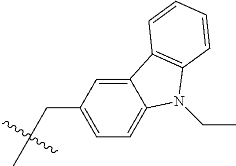 |
| A116 | 9-Ethyl-9H-carbazole-3-carboxylic acid {1-[3-(4-chloro-phenyl)- adamantan-1-yl]-ethyl}-amide | NH | CH₃ | 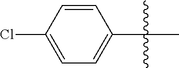 | 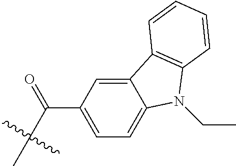 |
| A117 | 1-{1-[3-(4-Chloro-phenyl)- adamantan-1-yl]-ethyl}-3-(4-chloro- 3-trifluoromethyl-phenyl)-urea | NH | CH₃ | 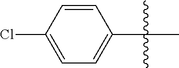 | 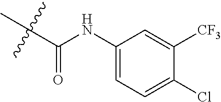 |
| A118 | 1-{1-[3-(4-Chloro-phenyl)- adamantan-1-yl]-ethyl}-3-(4-chloro- 3-trifluoromethyl-phenyl)-urea | NH | CH₃ | 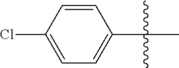 | 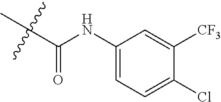 |
| A119 | (4-Bromo-thiophen-2-ylmethyl)-{1- [3-(4-chloro-phenyl)-adamantan-1- yl]-ethyl}-amine | NH | CH₃ | 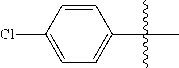 | 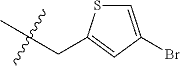 |
| A120 | {1-[3-(4-Chloro-phenyl)-adamantan- 1-yl]-ethyl}-(4-phenyl-thiophen-2- ylmethyl)-amine | NH | CH₃ | 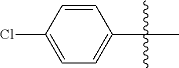 | 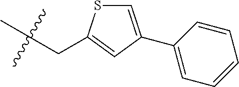 |

TABLE 2
Additional representative adamantane-based compounds.

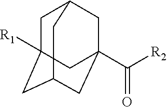

| Cmpd | Chemical name | R₁ | R₂ |
|---|---|---|---|
| A121 | 3-Phenyl-adamantane-1-carboxylic acid | 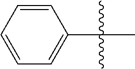 | OH |
| A122 | 3-(4-Fluoro-phenyl)-adamantane-1-carboxylic acid | 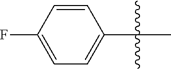 | OH |
| A123 | 3-(4-Chloro-phenyl)-adamantane-1-carboxylic acid | 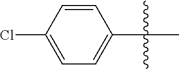 | OH |
| A124 | 1-Adamantan-1-yl-ethanone | H | CH₃ |
| A125 | 1-(3-Phenyl-adamantan-1-yl)-ethanone | 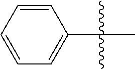 | CH₃ |
| A126 | 1-[3-(4-Fluoro-phenyl)-adamantan-1-yl]-ethanone | 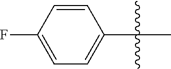 | CH₃ |
| A127 | 1-[3-(4-Chloro-phenyl)-adamantan-1-yl]-ethanone | 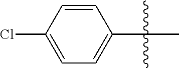 | CH₃ |
| A128 | 2-(Adamantane-1-carbonyl)-malonic acid dimethyl ester | H | 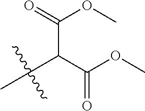 |
| A129 | 2-[3-(4-Chloro-phenyl)-adamantane-1-carbonyl]- malonic acid dimethyl ester | 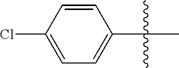 | 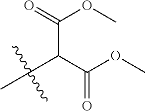 |
| A130 | 3-(4-Chloro-phenyl)-1-[3-(4-chloro-phenyl)- adamantan-1-yl]-propenone | 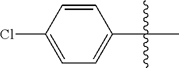 | 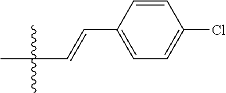 |
| A131 | 4-{3-[3-(4-Chloro-phenyl)-adamantan-1-yl]-3-oxo- propenyl}-benzonitrile | 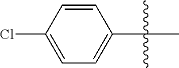 | 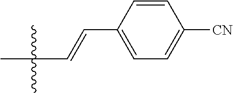 |
| A132 | 1-[3-(4-Chloro-phenyl)-adamantan-1-yl]-3-(4- hydroxy-phenyl)-propenone | 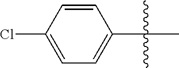 | 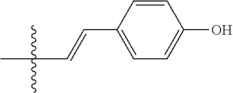 |
| A133 | 1-[3-(4-Chloro-phenyl)-adamantan-1-yl]-3- naphthalen-2-yl-propenone | 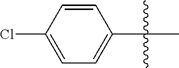 | 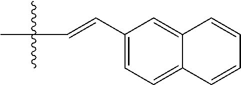 |
| A134 | 1-[3-(4-Chloro-phenyl)-adamantan-1-yl]-3-(6-chloro- pyridin-3-yl)-propenone | 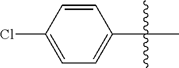 | 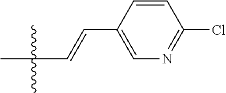 |
| A135 | 1-[3-(4-Chloro-phenyl)-adamantan-1-yl]-3-(1H- imidazol-4-yl)-propenone | 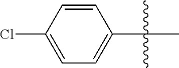 | 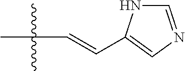 |
| A136 | 1-[3-(4-Chloro-phenyl)-adamantan-1-yl]-3-(9-ethyl- 9H-carbazol-3-yl)-propenone | 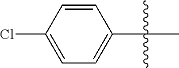 | 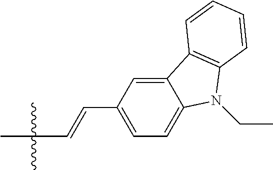 |
| A137 | 1-[3-(4-Chloro-phenyl)-admantan-1-yl]-3-(4- phenyl-thiophen-2-yl)-propenone | 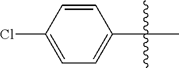 | 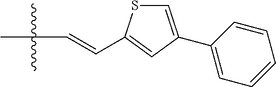 |

TABLE 3
Representative compounds

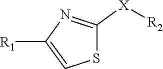

| # | X | R₁ | R₂ | Chemical name |
|---|---|---|---|---|
| B1 | 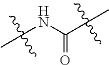 | 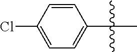 | 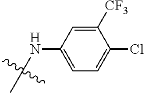 | 1-[4-(4-Chloro-phenyl)- thiazol-2-yl]-3-(4-chloro-3- trifluoromethyl-phenyl)-urea |
| B2 | 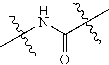 | 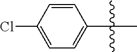 | 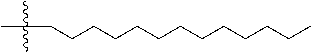 | Tetradecanoic acid [4-(4- chloro-phenyl)-thiazol-2-yl]- amide |
| B3 | 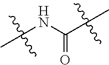 | 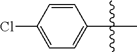 | 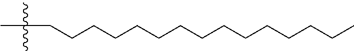 | Hexadecanoic acid [4-(4- chloro-phnyl)-thiazol-2-yl]- amide |
| B4 | 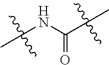 | 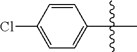 | 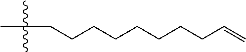 | Undec-10-enoic acid [4-(4- chloro-phenyl)-thiazol-2-yl]- amide |
| B5 | 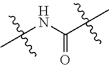 | 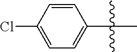 | 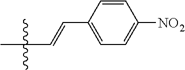 | N-[4-(4-Chloro-phenyl)- thiazol-2-yl]-3-(4-nitro- phenyl)-acrylamide |
| B6 | 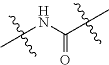 | 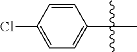 | 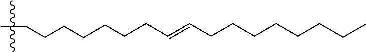 | Octadec-9-enoic acid [4-(4- chloro-phenyl)-thiazol-2-yl]- amide |
| B7 | 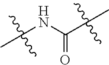 | 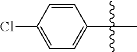 | 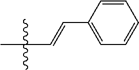 | N-[4-(4-Chloro-phenyl)- thiazol-2-yl]-3-phenyl- acrylamide |
| B8 | 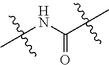 | 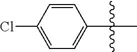 | 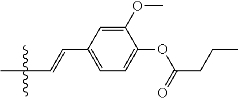 | Butyric acid 4-{2-[4-(4- chloro-phenyl)-thiazol-2- ylcarbamoyl]-vinyl}-2- methoxy-phenyl ester |
| B9 | 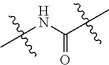 | 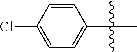 | 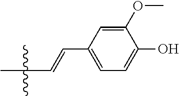 | N-[4-(3-Chloro-phenyl)- thiazol-2-yl]-3-(4-hydroxy- 3-methoxy-phenyl)- acrylamide |
| B10 | 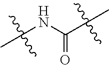 | 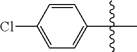 | 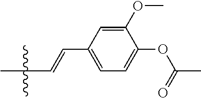 | Acetic acid 4-{2-[4-(4- chloro-phenyl)-thiazol- 2-ylcarbamoyl]-vinyl}-2- methoxy-phenyl ester |
| B11 | 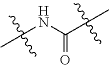 | 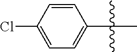 | 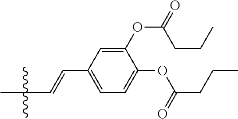 | Butyric acid 2-butyryloxy- 5-{2-[4-(4-chloro-phenyl)- thiazol-2-ylcarbamoyl]- vinyl}-phenyl ester |
| B12 | 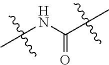 | 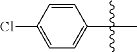 | 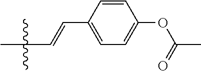 | Acetic acid 4-{2-[4-(4- chloro-phenyl)-thiazol-2- ylcarbamoyl]-vinyl}- phenyl ester |
| B13 | 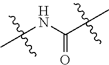 | 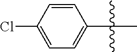 | 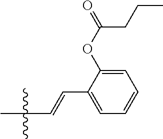 | Butyric acid 2-{2-[4-(4- chloro-phenyl)-thiazol-2- ylcarbamoyl]-vinyl}-phenyl ester |
| B14 | 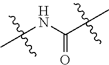 | 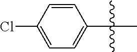 | 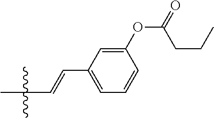 | Butyric acid 3-{2-[4-(4- chloro-phenyl)-thiazol-2- ylcarbamoyl]-vinyl}- phenyl ester |
| B15 | 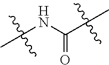 | 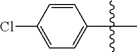 | 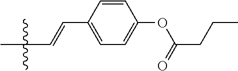 | Butyric acid 4-{2-[4-(4- chloro-phenyl)-thiazol-2- ylcarbamoyl]-vinyl}-phenyl ester |
| B16 | 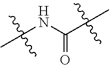 | 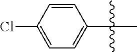 | 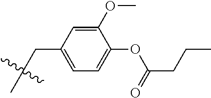 | Butyric acid 4-{[4-(4-chloro- phenyl)-thiazol-2- ylcarbamoyl]-methyl}-2- methoxy-phenyl ester |
| B17 | 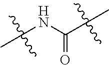 | 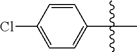 | 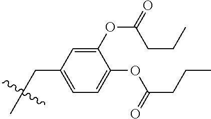 | Butyric acid 2-butyryloxy- 5-{[4-(4-chloro-phenyl)- thiazol-2-ylcarbamoyl]- methyl}-phenyl ester |
| B18 | 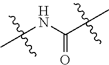 | 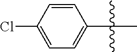 | 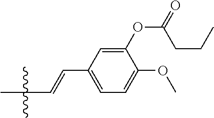 | Butyric acid 5-{2-[4-(4- chloro-phenyl)-thiazol-2- ylcarbamoyl]-vinyl}-2- methoxy-phenyl ester |
| B19 | 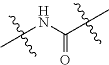 | 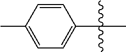 | 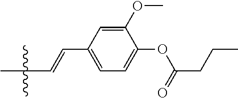 | Butyric acid 2-methoxy-4-[2- (4-p-tolyl-thiazol-2- ylcarbamoyl)-vinyl]-phenyl ester |
| B20 | 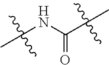 | 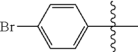 | 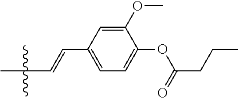 | Butyric acid 4-{2-[4-(4- bromo-phenyl)-thiazol-2- ylcarbamoyl]-vinyl}-2- methoxy-phenyl ester |
| B21 | 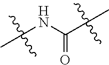 | 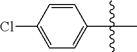 | 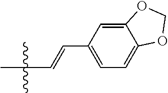 | 3-Benzo[1,3]dioxol-5-yl-N- [4-(4-chloro-phenyl)- thiazol-2-yl]-acrylamide |
| B22 | 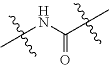 | 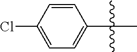 | 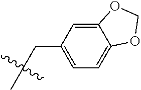 | 2-Benzo[1,3]dioxol-5-yl- N-[4-(4-chloro-phenyl)- thiazol-2-yl]-acetamide |
| B23 | 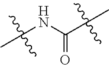 | 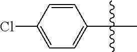 | 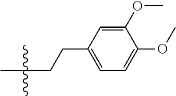 | N-[4-(4-Chloro-phenyl)- thiazol-2-yl]-3-(3,4- dimethoxy-phenyl)- propionamide |
| B24 | 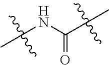 | 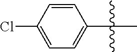 | 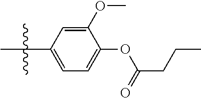 | Butyric acid 4-[4-(4-chloro- phenyl)-thiazol-2- ylcarbamoyl]-2-methoxy- phenyl ester |
| B25 | 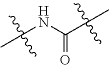 | 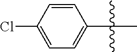 | 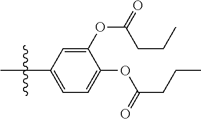 | Butyric acid 2-butyryloxy-4- [4-(4-chloro-phenyl)-thiazol- 2-ylcarbamoyl]-phenyl ester |
| B26 | 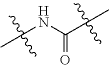 | 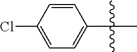 | 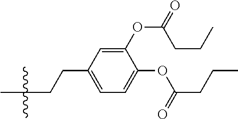 | Butyric acid 2-butyryloxy-4- {2-[4-(4-chloro-phenyl)- thiazol-2-ylcarbamoyl]- ethyl}-phenyl ester |
| B27 | 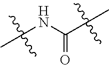 | 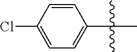 | 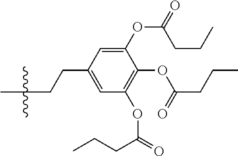 | Butyric acid 2,6-bis- butyryloxy-4-[4-(4-chloro- phenyl)-thiazol-2- ylcarbamoyl]-phenyl ester |
| B28 | 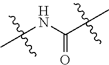 | 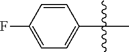 | 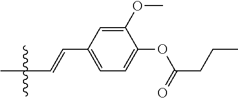 | Butyric acid 4-{2-[4-(4- fluoro-phenyl)-thiazol-2- ylcarbamoyl]-vinyl}-2- methoxy-phenyl ester |
| B29 | 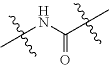 | 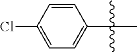 | 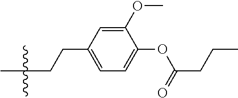 | Butyric acid 4-{2-[4-(4- chloro-phenyl)-thiazol-2- ylcarbamoyl]-ethyl}-2- methoxy-phenyl ester |
| B30 | 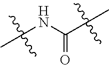 | 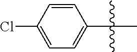 | 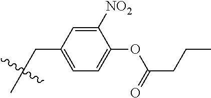 | Butyric acid 4-{[4-(4- chloro-phenyl)-thiazol-2- ylcarbamoyl]-methyl}-2- nitro-phenyl ester |
| B31 | 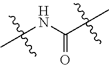 | 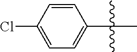 | 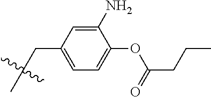 | Butyric acid 2-amino-4-{[4- (4-chloro-phenyl)-thiazol-2- ylcarbamoyl]-methyl}- phenyl ester |
| B32 | 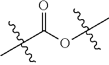 | 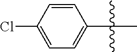 | —CH₂CH₃ | 4-(4-Chloro-phenyl)-thiazole- 2-carboxylic acid ethyl ester |
| B33 | 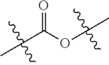 | 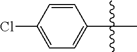 | H | 4-(4-Chloro-phenyl)-thiazole- 2-carboxylic acid |
| B34 | 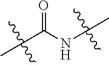 | 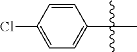 | 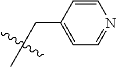 | 4-(4-Chloro-phenyl)thiazole- 2-carboxylic acid (pyridin- 4-ylmethyl)amide |
| B35 | 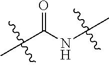 | 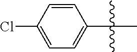 | 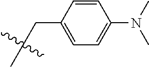 | 4-(4-Chloro-phenyl)-thiazole- 2-carboxylic acid 4- dimethylamino-benzylamide |
| B36 | 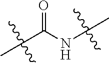 | 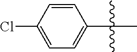 | 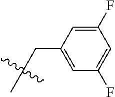 | 4-(4-Chloro-phenyl)-thiazole- 2-carboxylic acid 3,5- difluoro-benzylamide |
| B37 | 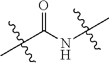 | 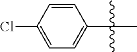 | 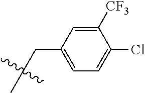 | 4-(4-Chloro-phenyl)-thiazole- 2-carboxylic acid 4-chloro-3- trifluoromethyl-benzylamide |
| B38 | 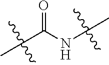 | 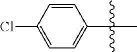 | 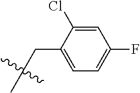 | 4-(4-Chloro-phenyl)-thiazole- 2-carboxylic acid 2-chloro-4- fluoro-benzylamide |
| B39 | 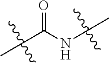 | 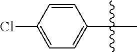 | 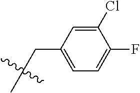 | 4-(4-Chloro-phenyl)-thiazole- 2-carboxylic acid 3-chloro-4- fluoro-benzylamide |
| B40 | 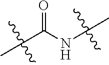 | 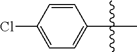 | 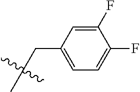 | 4-(4-Chloro-phenyl)-thiazole- 2-carboxylic acid 3,4- difluoro-benzylamide |
| B41 | 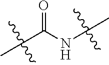 | 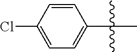 | 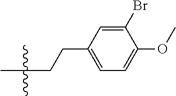 | 4-(4-Chloro-phenyl)-thiazole- 2-carboxylic acid [2-(3- bromo-4-methoxy-phenyl)- ethyl]-amide |
| B42 | 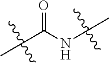 | 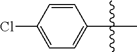 | 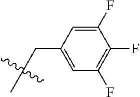 | 4-(4-Chloro-phenyl)-thiazole- 2-carboxylic acid 3,4,5- trifluoro-benzylamide |
| B43 | 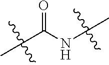 | 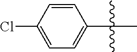 | 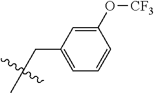 | 4-(4-Chloro-phenyl)-thiazole- 2-carboxylic acid 3- trifluoromethoxy- benzylamide |
| B44 | 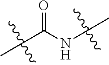 | 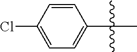 | 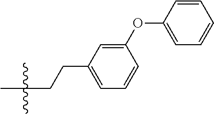 | 4-(4-Chloro-phenyl)-thiazole- 2-carboxylic acid [2-(3- phenoxy-phenyl)-ethyl]- amide |
| B45 | 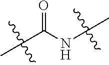 | 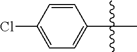 | 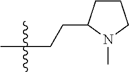 | 4-(4-Chloro-phenyl)-thiazole- 2-carboxylic acid [2-(1- methyl-pyrrolidin-2-yl)- ethyl]-amide |
| B46 | 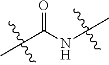 | 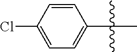 | 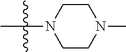 | 4-(4-Chloro-phenyl)-thiazole- 2-carboxylic acid (4-methyl- piperazin-1-yl)-amide |
| B47 | 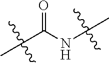 | 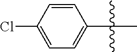 | 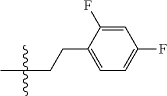 | N-[4-(4-Chloro-phenyl)- thiazol-2-yl]-3-(2,4-difluoro- phenyl)-propionamide |
| B48 | 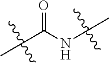 | 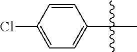 | 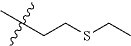 | 4-(4-Chloro-phenyl)-thiazole- 2-carboxylic acid (2- ethylsulfanyl-ethyl)-amide |
| B49 | 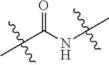 | 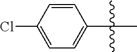 | 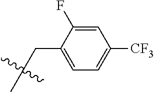 | 4-(4-Chloro-phenyl)-thiazole- 2-carboxylic acid 2-fluoro-4- trifluoromethyl-benzylamide |
| B50 | 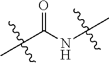 | 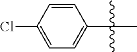 | 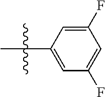 | 4-(4-Chloro-phenyl)-thiazole- 2-carboxylic acid (3,5- difluoro-phenyl)-amide |
| B51 | 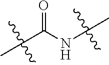 | 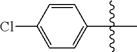 | 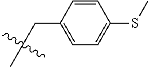 | 4-(4-Chloro-phenyl)-thiazole- 2-carboxylic acid 4- methylsulfanyl-benzylamide |
| B52 | 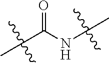 | 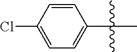 | 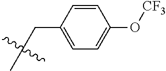 | 4-(4-Chloro-phenyl)-thiazole- 2-carboxylic acid 4- trifluoromethoxy- benzylamide |
| B53 | 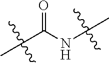 | 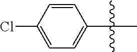 | 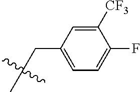 | 4-(4-Chloro-phenyl)-thiazole- 2-carboxylic acid 4-fluoro-3- trifluoromethyl-benzylamide |
| B54 | 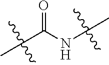 | 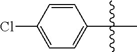 | 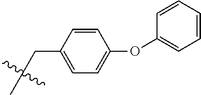 | 4-(4-Chloro-phenyl)-thiazole- 2-carboxylic acid 4-phenoxy benzylamide |
| B55 | 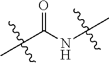 | 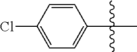 | 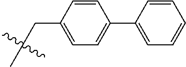 | 4-(4-Chloro-phenyl)-thiazole- 2-carboxylic acid (biphenyl- 4-ylmethyl)-amide |
| B56 | 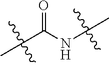 | 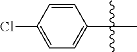 | 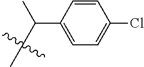 | 4-(4-Chloro-phenyl)-thiazole- 2-carboxylic acid [1-(4- chloro-phenyl)-ethyl]-amide |
| B57 | 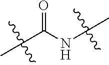 | 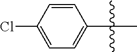 | 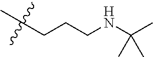 | 4-(4-Chloro-phenyl)-thiazole- 2-carboxylic acid (3-tert- butylamino-propyl)-amide |
| B58 | 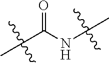 | 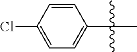 | 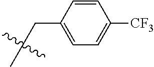 | 4-(4-Chloro-phenyl)-thiazole- 2-carboxylic acid 4- trifluoromethyl-benzylamide |
| B59 | 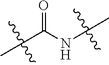 | 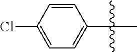 | 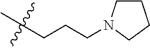 | 4-(4-Chloro-phenyl)-thiazole- 2-carboxylic acid (3- pyrrrolidin-1-yl-propyl)- amide |
| B60 | 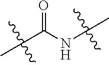 | 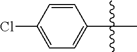 | 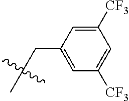 | 4-(4-Chloro-phenyl)-thiazole- 2-carboxylic acid 3,5-bis- trifluoromethyl-benzylamide |
| B61 | 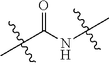 | 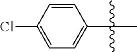 | 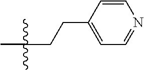 | 4-(4-Chloro-phenyl)-thiazole- 2-carboxylic acid (2- pyridin-4-yl-ethyl)-amide |
| B62 | 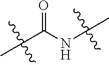 | 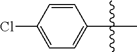 | 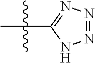 | 4-(4-Chloro-phenyl)-thiazole- 2-carboxylic acid (1H- tetrazol-5-yl)-amide |
| B63 | 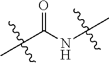 | 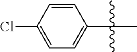 | 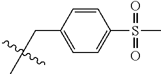 | 4-(4-Chloro-phenyl)-thiazole- 2-carboxylic acid 4- methanesulfonyl- benzylamide |
| B64 | 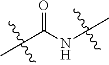 | 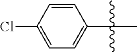 | 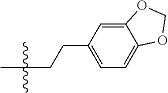 | 4-(4-Chloro-phenyl)-thiazole- 2-carboxylic acid (2- benzo[1,3]dioxol-5-yl-ethyl)- amide |
| B65 | 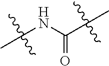 | 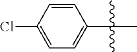 | 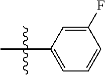 | N-[4-(4-Chloro-phenyl)- thiazol-2-yl]-3-fluoro- benzamide |
| B66 | 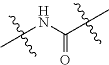 | 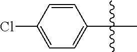 | 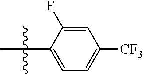 | N-[4-(4-Chloro-phenyl)- thiazol-2-yl]-2-fluoro-4- trifluoromethyl-benzamide |
| B67 | 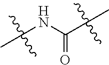 | 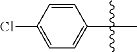 | 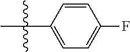 | N-[4-(4-Chloro-phenyl)- thiazol-2-yl]-4-fluoro- benzamide |
| B68 | 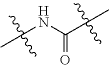 | 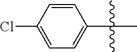 | 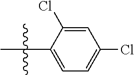 | 2,4-Dichloro-N-[4-(4-chloro- phenyl)-thiazol-2-yl]- benzamide |
| B69 | 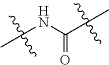 | 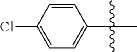 | 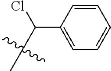 | 2-Chloro-N-[4-(4-chloro- phenyl)-thiazol-2-yl]-2- phenyl-acetamide |
| B70 | 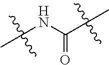 | 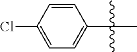 | 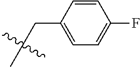 | N-[4-(4-Chloro-phenyl)- thiazol-2-yl]-2-(4-fluoro- phenyl)-acetamide |
| B71 | 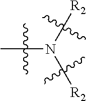 | 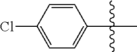 | 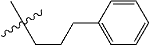 | [4-(4-Chloro-phenyl)-thiazol- 2-yl]-bis-(3-phenyl-propyl)- amine |
| B72 | 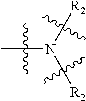 | 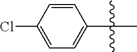 | 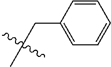 | Dibenzyl-[4-(4-chloro- phenyl)-thiazol-2-yl]-amine |
| B73 | 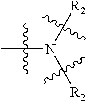 | 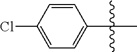 | 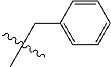 | Benzyl-[4-(4-chloro-phenyl)- thiazol-2-yl]-amine |
| B74 | 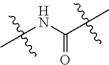 | 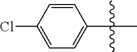 | 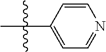 | 4-(4-Chloro-phenyl)-thiazole- 2-carboxylic acid (2- pyridin-4-yl)-amide |
| B75 | 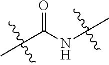 | 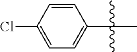 | 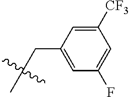 | 4-(4-Chloro-phenyl)-thiazole- 2-carboxylic acid 3-fluoro-5- trifluoromethyl-benzylamide |
| B76 | 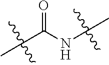 | 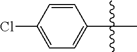 | 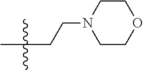 | 4-(4-Chloro-phenyl)-thiazole- 2-carboxylic acid (2- morpholin-4-yl-ethyl)-amide |
| B77 | 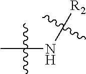 | 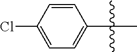 | 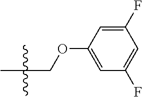 | [4-(4-Chloro-phenyl)-thiazol- 2-yl]-(3,5-difluoro- phenoxymethyl)-amine |
| B78 | 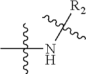 | 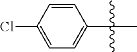 | 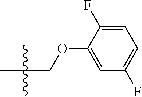 | [4-(4-Chloro-phenyl)-thiazol- 2-yl]-(2,5-difluoro- phenoxymethyl)-amine |
| B79 | 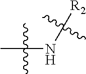 | 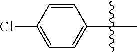 | 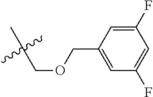 | [4-(4-Chloro-phenyl)-thiazol- 2-yl]-(3,5-difluoro- benzyloxymethyl)-amine |

TABLE 4
Additional representative compounds

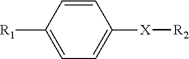

| # | X | R₁ | R₂ | Chemical name |
|---|---|---|---|---|
| B80 | 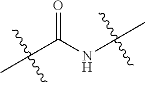 | 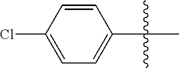 | 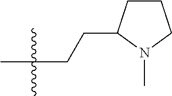 | 4′-Chloro-biphenyl-3-carboxylic acid [2-(1- methyl-pyrrolidin-2-yl)-ethyl]-amide |
| B81 | 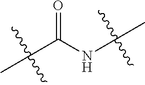 | 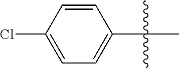 | 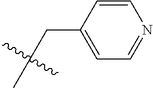 | 4′-Chloro-biphenyl-3-carboxylic acid (pyridin-4-ylmethyl)-amide |
| B82 | 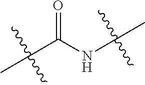 | 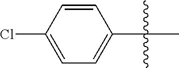 | 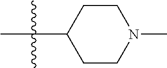 | 4′-Chloro-biphenyl-3-carboxylic acid (1- methyl-piperidin-4-yl)-amide |
| B83 | 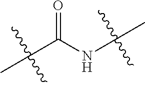 | 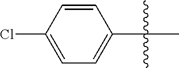 | 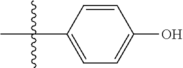 | 4′-Chloro-biphenyl-3-carboxylic acid (4- hydroxy-phenyl)-amide |
| B84 | 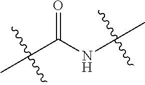 | 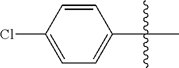 | 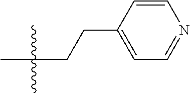 | 4′-Chloro-biphenyl-3-carboxylic acid (2- pyridin-4-yl-ethyl)-amide |
| B85 | 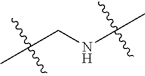 | 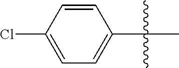 | 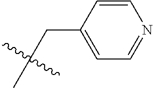 | (4′-Chloro-biphenyl-3-ylmethyl)-pyridin-4- ylmethyl-amine |
| B86 | 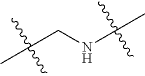 | 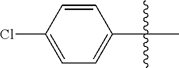 | 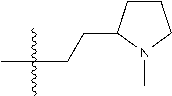 | (4′-Chloro-biphenyl-3-ylmethyl)-[2-(1- methyl-pyrrolidin-2-yl)-ethyl]-amine |

TABLE 5
Additional representative compounds

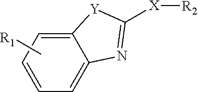

| # | X | Y | R₁ | R₂ | Chemical name |
|---|---|---|---|---|---|
| B87 | 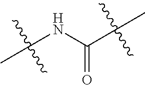 | O | 5-Chloro- | 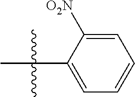 | N-(5-Chloro- benzooxazol-2-yl)-2- nitro-benzamide |
| B88 | 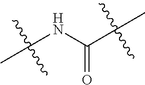 | O | 5-Chloro | 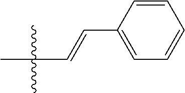 | N-(5-Chloro- benzooxazol-2-yl)-3- phenyl-acrylamide |
| B89 | 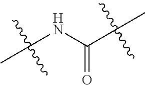 | O | 5-Chloro | 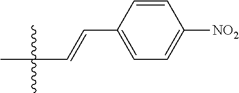 | N-(5-Chloro- benzooxazol-2-yl)-3- (4-nitro-phenyl)- acrylamide |
| B90 | 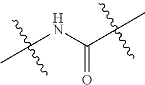 | O | 5-Chloro- | 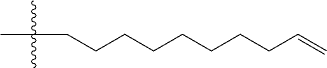 | Undec-10-enoic acid (5-chloro-benzooxazol- 2-yl)-amide |
| B91 | 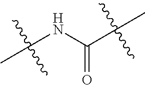 | O | 5-Chloro- | 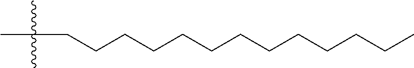 | Tetradecanoic acid (5- chloro-benzooxazol-2- yl)-amide |
| B92 | 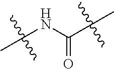 | O | 5-Chloro- | 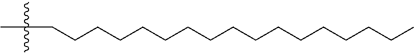 | Hexadecanoic acid (5- chloro-benzooxazol-2- yl)-amide |
| B93 | 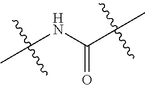 | O | 5-Chloro- | 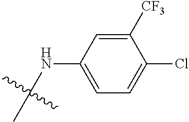 | 1-(5-Chloro- benzooxazol-2-yl)-3- (4-chloro-3- trifluoromethyl- phenyl)-urea |
| B94 | 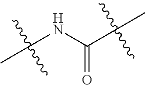 | S | H— | 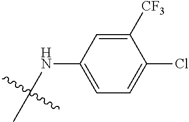 | 1-Benzothiazol-2-yl-3- (4-chloro-3- trifluoromethyl- phenyl)-urea |
| B95 | 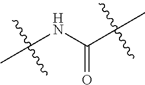 | S | 5-Chloro- | 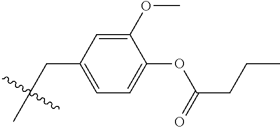 | Butyric acid 4-[(6- chloro-benzothiazol-2- ylcarbamoyl)-methyl]- 2-methoxy-phenyl ester |
| B96 | 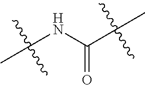 | S | H— | 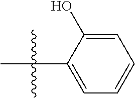 | N-(5-Chloro- benzothiazol-2-yl)-2- hydroxy-benzamide |
| B97 | 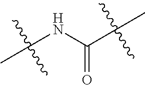 | O | 5-Chloro- | 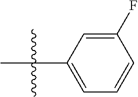 | N-(5-Chloro- benzooxazol-2-yl)-3- fluoro-benzamide |

### Example 15

The sphingosine kinase inhibition activities of representative compounds of Example 14 are presented below. Human SK was incubated with 6 μg/mL of the indicated compounds, and then assayed for activity as described above. Values in the column labeled “Recombinant SK (% inhibition)” represent the percentage of SK activity that was inhibited. MDA-MB-231 cells were incubated with 20 μg/mL of the indicated compounds and then assayed for endogenous SK activity as indicated above. Values in the column labeled “Cellular S1P (% inhibition)” represent the percentage of S1P production that was inhibited. Additionally, MDA-MB-231 cells were treated with varying concentration of certain compounds and the amount of S1P produced by the cells was determined. Values in the column labeled “Cellular S1P IC₅₀ (μM)” represent the concentration of compound required to inhibit the production of S1P by 50%. ND=not determined.

TABLE 6
SK inhibition data.

| | Recombinant | | Cellular |
|---|---|---|---|
| | SK | Cellular S1P | S1P |
| Compound | (% inhibition) | (% inhibition) | IC₅₀ (μM) |
| A1 | 38 | 0 | ND |
| A2 | 0 | ND | ND |
| A3 | 6 | ND | ND |
| A4 | 44 | 14 | ND |
| A5 | 100 | 17 | ND |
| A6 | 72 | 90 | 15 |
| A7 | 100 | 96 | ND |
| A8 | 49 | 0 | ND |
| A9 | 84 | 40 | ND |
| A10 | 3 | 9 | ND |
| A11 | 17 | 0 | ND |
| A12 | 36 | 3 | ND |
| A13 | 78 | 0 | ND |
| A14 | 19 | ND | ND |
| A16 | 8 | ND | ND |
| A17 | 56 | ND | ND |
| A18 | 0 | ND | ND |
| A20 | 0 | ND | ND |
| A21 | 65 | ND | ND |
| A22 | 56 | ND | ND |
| A23 | 0 | ND | ND |
| A24 | 20 | ND | ND |
| A25 | 47 | ND | ND |
| A26 | 36 | ND | ND |
| A27 | 50 | ND | ND |
| A28 | 6 | ND | ND |
| A29 | 55 | 0 | ND |
| A30 | 1 | ND | ND |
| A31 | 74 | ND | ND |
| A32 | 17 | ND | ND |
| A33 | 10 | ND | ND |
| A34 | 0 | ND | ND |
| A35 | 87 | 0 | ND |
| A36 | 37 | 72 | ND |
| A37 | 24 | 36 | ND |
| A38 | 40 | 34 | ND |
| A39 | 19 | 26 | ND |
| A40 | 100 | 52 | ND |
| A41 | 67 | 23 | ND |
| A42 | 5 | ND | ND |
| A43 | 0 | 0 | ND |
| A44 | 33 | 88 | 35 |
| A45 | 64 | ND | ND |
| A46 | 4 | ND | ND |
| A47 | 26 | ND | ND |
| A48 | 36 | 14 | ND |
| A49 | 33 | ND | ND |
| A50 | 0 | 44 | ND |
| A51 | 84 | 88 | 25 |
| A52 | 54 | 61 | ND |
| A53 | 52 | ND | ND |
| A54 | 95 | ND | ND |
| A55 | 8 | ND | ND |
| A56 | 33 | 40 | ND |
| A57 | 30 | 83 | 60 |
| A58 | 67 | 55 | ND |
| A59 | 0 | ND | ND |
| A60 | 0 | 23 | ND |
| A61 | 58 | 24 | ND |
| A62 | 13 | 92 | 26 |
| A63 | 0 | 39 | ND |
| A64 | 41 | 80 | 63 |
| A65 | 3 | ND | ND |
| A66 | 92 | 8 | ND |
| A67 | 10 | ND | ND |
| A68 | 17 | 0 | ND |
| A69 | 40 | 13 | ND |
| A70 | 33 | 4 | ND |
| A71 | 27 | 0 | ND |
| A72 | 14 | 1 | ND |
| A73 | 53 | ND | ND |
| A74 | 0 | 28 | ND |
| A75 | ND | 41 | ND |
| A76 | 42 | ND | ND |
| A77 | 27 | ND | ND |
| A78 | 43 | ND | ND |
| A79 | 19 | ND | ND |
| A80 | 5 | ND | ND |
| A81 | 67 | ND | ND |
| A82 | 75 | ND | ND |
| A83 | 60 | 88 | 16 |
| A84 | 84 | ND | ND |
| A85 | 0 | ND | ND |
| A86 | 6 | ND | ND |
| A87 | 75 | 55 | 64 |
| A88 | 1 | ND | ND |
| A89 | 37 | 1 | ND |
| A90 | 26 | 16 | ND |
| A93 | 70 | ND | ND |
| A104 | 0 | ND | ND |
| A114 | 33 | 46 | 51 |
| A118 | 77 | 5 | ND |
| A130 | 38 | ND | ND |
| A131 | 41 | ND | ND |
| A132 | 8 | ND | ND |
| A133 | 36 | ND | ND |
| A134 | 52 | ND | ND |
| A135 | 64 | ND | ND |
| B1 | ND | 32 | ND |
| B2 | 30 | 43 | ND |
| B3 | 88 | 34 | ND |
| B4 | 53 | 39 | ND |
| B5 | 0 | 25 | ND |
| B6 | 0 | 21 | ND |
| B7 | 71 | 21 | ND |
| B8 | ND | 80 | 34 |
| B9 | ND | 32 | ND |
| B10 | 100 | 48 | ND |
| B11 | ND | 55 | ND |
| B12 | ND | 13 | ND |
| B13 | 0 | 0 | ND |
| B14 | 0 | 39 | ND |
| B15 | 73 | 23 | ND |
| B16 | ND | 83 | ND |
| B17 | ND | 57 | ND |
| B18 | ND | 65 | ND |
| B19 | 36 | 53 | ND |
| B20 | 6 | 62 | ND |
| B21 | 26 | 41 | ND |
| B22 | 34 | 33 | ND |
| B23 | 45 | 14 | ND |
| B24 | 0 | 69 | ND |
| B25 | 0 | 79 | ND |
| B26 | 0 | 79 | ND |
| B27 | ND | 68 | ND |
| B28 | 87 | 65 | ND |
| B29 | 0 | ND | ND |
| B30 | 0 | ND | ND |
| B31 | 58 | ND | ND |
| B32 | ND | ND | ND |
| B33 | ND | ND | ND |
| B34 | 0 | 28 | ND |
| B35 | 80 | 17 | ND |
| B36 | 14 | 0 | ND |
| B37 | 23 | 0 | ND |
| B38 | 75 | 0 | ND |
| B39 | 69 | 0 | ND |
| B40 | 56 | 0 | ND |
| B41 | 22 | 0 | ND |
| B42 | 79 | 0 | ND |
| B43 | 59 | 0 | ND |
| B44 | 69 | 0 | ND |
| B45 | 42 | 0 | ND |
| B46 | 80 | 0 | ND |
| B47 | 21 | ND | ND |
| B48 | 56 | ND | ND |
| B49 | 67 | ND | ND |
| B50 | 21 | ND | ND |
| B51 | 36 | ND | ND |
| B52 | 78 | ND | ND |
| B53 | 44 | ND | ND |
| B54 | 25 | ND | ND |
| B55 | 20 | ND | ND |
| B56 | 81 | ND | ND |
| B57 | 16 | ND | ND |
| B58 | 86 | ND | ND |
| B59 | 46 | ND | ND |
| B60 | 87 | ND | ND |
| B61 | 0 | ND | ND |
| B62 | 60 | ND | ND |
| B63 | 3 | ND | ND |
| B64 | 90 | ND | ND |
| B65 | 66 | ND | ND |
| B66 | 61 | ND | ND |
| B67 | 41 | ND | ND |
| B68 | 73 | ND | ND |
| B69 | 55 | ND | ND |
| B70 | 54 | ND | ND |
| B71 | 44 | 15 | ND |
| B72 | 79 | 27 | ND |
| B76 | ND | 81 | 5 |
| B74 | ND | ND | ND |
| B75 | 3 | ND | ND |
| B76 | 51 | ND | ND |
| B77 | 85 | ND | ND |
| B78 | 70 | ND | ND |
| B79 | 53 | ND | ND |
| B80 | ND | 70 | ND |
| B81 | ND | 14 | ND |
| B82 | ND | 67 | ND |
| B83 | ND | 55 | ND |
| B84 | ND | 76 | ND |
| B85 | ND | ND | ND |
| B86 | ND | ND | ND |
| B87 | ND | 64 | 22 |
| B88 | ND | 46 | ND |
| B89 | ND | 74 | 5.8 |
| B90 | ND | 39 | ND |
| B91 | ND | 0 | ND |
| B92 | ND | 4 | ND |
| B93 | ND | 53 | ND |
| B94 | ND | 13 | ND |
| B95 | ND | ND | ND |
| B96 | ND | 18 | ND |
| B97 | 71 | 38 | ND |

### METHODS

Ischemia-reperfusion of the kidney (mild model): Male C57/Bl6 mice (approximately 24 g) were first dosed with ABC294640 (50 mg/kg in 0.1 ml by oral gavage) or vehicle (0.1 ml in 0.375% Tween 80 in Phosphate-Buffered Saline), immediately followed by intraperitoneal injection of ketamine/xylazine for anesthesia. The procedure was performed on a heated surface using homeothermic pads to ensure the maintenance of animal body temperature. A midline incision was made and the two renal pedicles were located and clamped for 22 minutes or 25 minutes as indicated. Total blockage of the renal pedicle and thus artery was confirmed after several minutes as the kidney were seen to be dark red to purple in color, assuring correct clamp placement. After the scheduled time elapsed, the clamps were removed and the kidneys were observed to ensure reperfusion as indicated by returning to their original color. One milliliter of pre-warmed (37° C.) sterile saline was instilled into the peritoneum at the time of closing using sutures for musculature and wound clips for the skin incision. Animals were maintained on homethermic pads until awakening from anesthesia and post-operatively assessed for health.

Ischemia-reperfusion of the kidney (severe model): The irreversible model was performed in a similar manner to the reversible model described above with the exception that the right kidney pedicle was tied off and the kidney removed and the left kidney was clamped for 45 minutes.

Statistical Analysis: Survival rates were compared by the log-rank test. For other parameters, we used the Student's t-test to compare values of 2 groups. Differences are considered significant when p<0.05.

Liver transplantation: Inbred male Lewis rats (200-250 g) were used to prevent immunological interference. Under ether anesthesia, heparin (200 IU) in 0.5 mL of lactated Ringer's solution was injected into the subhepatic vena cava. A 4-mm long stent prepared from polyethylene tubing (PE50) was inserted into the common bile duct and secured with a 6-0 suture. Livers were then flushed with 5 ml of ice-cold UW cold storage solution. Venous cuffs prepared from 14-gauge i.v. catheters were placed in the subhepatic vena cava and the portal vein. Liver explants were stored in UW solution (0-1° C.) for 4-24 h, rinsed with lactated Ringer's solution and then implanted (n=10 in each group). For implantation, livers of recipients were removed, and grafts were implanted by connecting the suprahepatic vena cava with a running suture. Cuffs were then inserted into the appropriate vessels and secured with 6-0 silk suture. The hepatic artery and bile duct were then anatomized with intraluminal stents. During implantation the portal vein was clamped for 18-20 minutes. Survival was assumed to be permanent when rats remain alive for 30 days after surgery. All animals received humane care in compliance with institutional guidelines.

Histology: Under pentobarbital anesthesia (50 mg/kg, i.p.), livers were rinsed with 10 ml normal saline and perfusion-fixed with 4% paraformaldehyde in phosphate buffer, embedded in paraffin, and sections were stained with hematoxylin-eosin (H+E). Necrotic areas in sections were quantified by image analysis using an Image-1/AT image acquisition and analysis system (Universal Imaging Corp., West Chester, Pa.) incorporating an Axioskop 50 microscope (Carl Zeiss, Inc., Thornwood, NY) and a 10× objective lens.

To detect steatosis, some liver grafts were frozen-sectioned after imbedded in Tissue-Tek OCT Compound. Fat droplets were visualized by Oil-Red-O staining. Relative areas in sections stained for lipids by Oil-Red-O were quantified by image analysis for the area with red color of lipid divided by the total cellular area.

Immunohistochemistry: Sections were deparaffinized in xylene, rehydrated in a series of graded alcohol concentrations and placed in phosphate buffered saline with 0.1% Tween-20. Immunohistochemical staining were performed using primary antibodies specific for SK, MPO, ED-1 and ICAM-1 at concentrations of 1:200-500 with 1% bovine albumin in PBS as appropriate. Appropriate peroxidase-conjugated secondary antibodies (DAKO Corp.) were then applied, followed by 3,3′-diaminobenzidine chromagen as the peroxidase substrate. A light counterstain of Meyer's hematoxylin was applied.

The TUNEL assay was performed to assess apoptosis using an In Situ Cell Death Detection Kit (Roche Diagnostics Corp., Indianapolis, Ind.). TUNEL-positive and negative cells were counted in 10 randomly selected fields using a 40× objective lens. Apoptosis was verified morphologically by identifying condensed and fragmented nuclei in 10 randomly selected fields in H+E slides (Grasl-Kraupp, Ruttkay-Nedecky et al. 1995).

Clinical chemistry: Blood samples were collected from the vena cava at various times, and ALT and bilirubin were measured enzymatically using analytic kits from Pointe Scientific.

Western blotting: Briefly, liver tissue was homogenized in a 0.1% Triton-X100 buffer containing a protease inhibitor cocktail, and the extract was centrifuged at 12,000×g for 10 min at 4° C. Cytosolic extracts (10-50 μg) was separated onto 10-16% SDS-PAGE gels, transferred onto nitrocellulose membranes using a semi-dry transfer technique and immunoblotted with primary antibodies specific for the proteins of interest. Horseradish peroxidase-conjugated secondary antibodies were applied, and detection was by chemiluminescence (ECL, Amersham). Expression of the protein of interest was quantified by the density of Western blot images by densitometry and standardized by house keeping gene actin.

Cytokine detection: Blood (500 μl) was collected into 150 μl of protease inhibitor aprotinin (Sigma). The serum was stored at −80° C. TNFα and IL-6 in sera were measured using commercially available enzyme-linked immunosorbent assay kits (Biosource, California) (Ikejima, Iimuro et al. 1996; Asakura, Ohkohchi et al. 2000).

Intravital Multiphoton microscopy: Under pentobarbital (50 mg/kg, i.p.) anesthesia, Rh123 (6 μmol/rat) and PI (0.12 μmol/rat) were infused into carotid artery at various times after LT and imaged by intravital multiphoton microscopy to evaluate mitochondrial polarization and cell death. Mice were intubated and ventilated with a small animal respirator. During collection of images, ventilation was briefly stopped to minimize movement artifacts. Calcein-AM (3 mg/rat) was infused into the rectal vein. Bromosulfophthalein (18 μmol/rat) was injected into the rectal vein 5 min before Calcein-AM to prevent its biliary excretion.

Imaging of fluorescent probes Rh123, PI and calcein in vivo was achieved using a Zeiss LSM 510 laser scanning multiphoton microscope system using IR excitation of 800-900 nm from a Coherent Chameleon tunable Ti-Sapphire femtosecond pulsed laser, which excites both red- and green-fluorescing (rhodamine- and fluorescein-like) fluorophores. For calcein fluorescence, excitation of 720-nm was used.

Quantitative real-time PCR (qPCR): Total RNA in liver homogenates was isolated using a QIAGEN RNeasy kit and quantified using a NanoDrop ND-1000 Spectrophotometer. cDNAs of mRNA of interest were generated using the Bio-Rad iScript cDNA Synthesis kit. qPCR was performed on a BioRAD MyiQ single-color real-time PCR detection system. The primers used for each gene were designed using Primer 3 software. PCR reactions were performed in a 96-well plate with a reaction mixture containing 15 μl iQ SYBR Green Supermix (Bio-Rad), cDNA template, and 200 nM each of forward and reverse primers in a total volume of 30 μL. All reactions were performed in triplicate. The thermal cycling conditions were; 95° C. for 3 min, followed by 40 cycles of 2-step amplification (10 sec at 95° C. and 45 sec at 57° C.). Data were analyzed with MyiQ software. The abundance of mRNA of interest was normalized against 18S rRNA, using the ΔΔCt method.

Statistical Analysis: Survival rates were compared by the log-rank (Mantel-Cox) and Gehan-Breslow-Wilcoxon tests with 10 rats in each group using GraphPad Prism 5. For other parameters, we used the Student's t-test to compare values of 2 groups and the ANOVA plus Student-Newman-Keuls post-hoc test to compare values of more than 2 groups. Differences are considered significant when p<0.05.

1. A method for preventing or treating ischemia-reperfusion injury in a mammal, comprising delivering to the mammal a sphingosine kinase inhibitor or a pharmaceutical composition containing a sphingosine kinase inhibitor.

2. The method according to claim 1 wherein the ischemia-reperfusion injury is due to a surgical procedure.

3. The method according to claim 2, wherein the surgical procedure is cardiac bypass surgery, aortic aneurysm repair or organ transplant.

4. The method according to claim 1 wherein the ischemia-reperfusion injury is due to hemorrhagic shock.

5. The method according to claim 1 wherein the ischemia-reperfusion injury is due to trauma.

6. The method according to claim 1 wherein the ischemia-reperfusion injury is due to a stroke resulting from cerebral infarction, cerebral hemorrhage, subarachnoid hemorrhage, or transient cerebral ischemia.

7. The method according to claim 1 wherein the ischemia-reperfusion injury is due to a myocardial infarction.

8. The method according to claim 1 wherein the ischemia-reperfusion injury is due to sepsis.

9. The method according to claim 1 wherein the ischemia-reperfusion injury is due to hypotension.

10. The method according to claim 1 wherein the ischemia-reperfusion injury occurs in the kidney.

11. The method according to claim 1 wherein the ischemia-reperfusion injury occurs in the brain.

12. The method according to claim 1 wherein the ischemia-reperfusion injury occurs in the heart.

13. The method according to claim 1 wherein the ischemia-reperfusion injury occurs in the liver.

14. The method according to claim 1, further comprising delivering to the mammal one or more therapeutic drugs effective in the treatment of ischemia-reperfusion injury.

15. The method according to claim 1 wherein the sphingosine kinase inhibitor is 3-(4-chlorophenyl)-N-(pyridinyl-4-methyl)adamantane-1-carboxamide or a pharmaceutically acceptable salt thereof.

16. The method according to claim 1, wherein the sphingosine kinase inhibitor is 3-(4-chlorophenyl)-N-(2-(3,4-dihydroxyphenyl)ethyl)adamantane-1-carboxamide or a pharmaceutically acceptable salt thereof.

17. The method according to claim 1, wherein the sphingosine kinase inhibitor is safingol; N,N-dimethylsphingosine; 5-naphthalen-2-yl-2H-pyrazole-3-carboxylic acid; 2-hydroxy-naphthalen-1-ylmethylene)-hydrazide; 2-(p-hydroxyanilino)-4-(p-chlorophenyl)thiazole; 5-(2,4-dihydroxy-benzylidene)-3-(4-methoxy-phenyl)-2-thioxo-thiazolidin-4-one; 2-(3,4-dihydroxy-benzylidene)-benzo[b]thiophen-3-one; 2-(3,4-dihydroxy-benzylidene)-benzofuran-3-one; B-5354a, b, or c; F-12509A; or S-15183 a or b.

18. The method according to claim 1, wherein the sphingosine kinase inhibitor is a compound having structural formula (I): 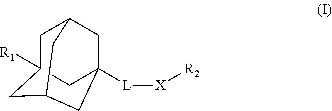
or a pharmaceutically acceptable salts thereof, wherein
L is a bond or is —C(R₃,R₄)—;
X is —C(R₃,R₄)N(R₅)—, —C(O)N(R₄)—, —N(R₄)C(O)—, —C(R₄,R₅)—, —N(R₄)—, —O—, —S—, —C(O)— —S(O)₂—, —S(O)₂N(R₄)— or —N(R₄)S(O)₂—;
R₁ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, mono or dialkylthiocarbamoyl, alkyl-S-alkyl, -heteroaryl-aryl, -alkyl-heteroaryl-aryl, —C(O)—NH-aryl, -alkenyl-heteroaryl, —C(O)-heteroaryl, or -alkenyl-heteroaryl-aryl;
R₃ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
wherein the alkyl and ring portion of each of the above R₁, R₂, and R₃ groups is optionally substituted with up to 5 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or NR′R″, wherein R′ and R″ are independently H or (C₁-C₆) alkyl, and wherein each alkyl portion of a substituent is optionally further substituted with 1, 2, or 3 groups independently selected from halogen, CN, OH, NH₂; and
R₄ and R₅ are independently H or alkyl, provided that when R₃ and R₄ are on the same carbon, and R₃ is oxo, then R₄ is absent.

19. The method according to claim 1, wherein the sphingosine kinase inhibitor is a compound having structural formula (III): 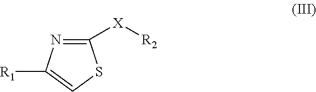
or a pharmaceutically acceptable salt thereof, wherein
X is —C(R₃,R₄)N(R₅)—, —C(O)N(R₄)—, —N(R₄)C(O)—, —C(R₄,R₅)—, —N(R₄)—, —O—, —S—, —C(O)— —S(O)₂—, —S(O)₂N(R₄)— or —N(R₄)S(O)₂—;
R₁ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
wherein the alkyl and ring portion of each of the above R₁ and R₂ groups is optionally substituted with up to 5 groups that are independently (C₁-C₆) alkyl, halogen, haloalkyl, —OC(O)(C₁-C₆ alkyl), —C(O)O(C₁-C₆ alkyl), —CONR′R″, —OC(O)NR′R″, —NR′C(O)R″, —CF₃, —OCF₃, —OH, C₁-C₆ alkoxy, hydroxyalkyl, —CN, —CO₂H, —SH, —S-alkyl, —SOR′R″, —SO₂R′, —NO₂, or NR′R″, wherein R′ and R″ are independently H or (C₁-C₆) alkyl, and wherein each alkyl portion of a substituent is optionally further substituted with 1, 2, or 3 groups independently selected from halogen, CN, OH, NH₂;
R₃ is H, alkyl, preferably lower alkyl, or oxo, provided that when R₃ and R₄ are on the same carbon, and R₃ is oxo, then R₄ is absent; and
R₄ and R₅ are independently H or alkyl, preferably lower alkyl.

20. The method according to claim 1, wherein the sphingosine kinase inhibitor is a compound having structural formula (IV): 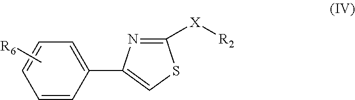
or a pharmaceutically acceptable salt thereof, wherein:
X is —C(R₃,R₄)N(R₅)—, —C(O)N(R₄)—, —N(R₄)C(O)—, —C(R₄,R₅)—, —N(R₄)—, —O—, —S—, —C(O)— —S(O)₂—, —S(O)₂N(R₄)— or —N(R₄)S(O)₂—;
R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅;
R₃ is H, alkyl or oxo, provided that when R₃ and R₄ are on the same carbon, and R₃ is oxo, then R₄ is absent;
R₄ and R₅ are independently H or (C₁-C₆)alkyl; and
R₆ is halogen, haloalkyl, alkoxy, haloalkoxy, hydroxyalkyl, alkanoyl, —COOH, —OH, —SH, —S-alkyl, —CN, —NO₂, or —NH₂.

21. The method according to claim 1, wherein the sphingosine kinase inhibitor is a compound having structural formula (V): 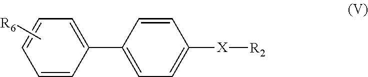
or a pharmaceutically acceptable salt thereof, wherein:
X is —C(R₃,R₄)N(R₅)—, —C(O)N(R₄)—, —N(R₄)C(O)—, —C(R₄,R₅)—, —N(R₄)—, —O—, —S—, —C(O)— —S(O)₂—, —S(O)₂N(R₄)— or —N(R₄)S(O)₂—;
R₁ is halogen, haloalkyl, alkoxy, haloalkoxy, hydroxyalkyl, alkanoyl, —COOH, —OH, —SH, —S-alkyl, —CN, —NO₂, or —NH₂:
R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and
R₃ is H, alkyl, preferably lower alkyl, or oxo, provided that when R₃ and R₄ are on the same carbon, and R₃ is oxo, then R₄ is absent; and
R₄ and R₅ are independently H or (C₁-C₆)alkyl.

22. The method according to claim 1, wherein the sphingosine kinase inhibitor is a compound having structural formula (VI): 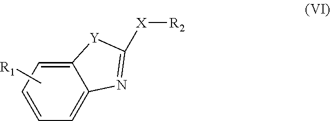
or a pharmaceutically acceptable salt thereof, wherein:
X is —C(R₃,R₄)N(R₅)—, —C(O)N(R₄)—, —N(R₄)C(O)—, —C(R₄,R₅)—, —N(R₄)—, —O—, —S—, —C(O)— —S(O)₂—, —S(O)₂N(R₄)— or —N(R₄)S(O)₂—;
Y is O or S;
R₁ is halogen, haloalkyl, alkoxy, haloalkoxy, hydroxyalkyl, alkanoyl, —COOH, —OH, —SH, —S-alkyl, —CN, —NO₂, or —NH₂;
R₂ is H, alkyl, cycloalkyl, cycloalkylalkyl, alkenyl, alkynyl, heteroalkyl, aryl, alkylaryl, alkenylaryl, heterocyclyl, heteroaryl, alkylheteroaryl, heterocycloalkyl, alkyl-heterocycloalkyl, acyl, aroyl, halogen, haloalkyl, alkoxy, haloalkoxy, hydroxyalkyl, alkanoyl, oxo (═O), —COOH, —OH, —SH, —S-alkyl, —CN, —NO₂, —NH₂, —CO₂(alkyl), —OC(O)alkyl, carbamoyl, mono or dialkylaminocarbamoyl, mono or dialkylcarbamoyl, mono or dialkylamino, aminoalkyl, mono- or dialkylaminoalkyl, thiocarbamoyl, or mono or dialkylthiocarbamoyl;
wherein the alkyl and ring portion of each of the above is optionally substituted with up to 5 groups that are independently (C₁-C₆) alkyl, halogen, haloalkyl, —OC(O)(C₁-C₆ alkyl), —C(O)O(C₁-C₆ alkyl), —CONR₄R₅, —OC(O)NR₄R₅, —NR₄C(O)R₅, —CF₃, —OCF₃, —OH, C₁-C₆ alkoxy, hydroxyalkyl, —CN, —CO₂H, —SH, —S-alkyl, —SOR₄R₅, —SO₂R₄R₅, —NO₂, or NR₄R₅; and
R₃ is H, alkyl, preferably lower alkyl, or oxo, provided that when R₃ and R₄ are on the same carbon, and R₃ is oxo, then R₄ is absent; and
R₄ and R₅ are independently H or (C₁-C₆)alkyl.
